(19) United States
(12) Patent Application Publication
(10) Pub. No.: US 2026/0090977 A1
(43) Pub. Date: Apr. 2, 2026

(54) WATER-IN-OIL COSMETIC COMPOSITION

(71) Applicant: Shin-Etsu Chemical Co., Ltd., Tokyo (JP)

(72) Inventors: Masaru MIYAUCHI, Tokyo (JP); Masayuki KONISHI, Tokyo (JP); Yoshinori INOKUCHI, Annaka-shi, Gunma (JP); Ryuji HORIGUCHI, Annaka-shi, Gunma (JP)

(21) Appl. No.: 19/111,053

(22) PCT Filed: Sep. 7, 2023
(86) PCT No.: PCT/JP2023/032676

§ 371 Date: Mar. 12, 2025

(30) Foreign Application Priority Data
Sep. 13, 2022 (JP) 2022-145584

(51) Int. Cl. A61K 8/894; A61K 8/06; A61K 8/31; A61K 8/37; A61Q 1/02

(52) U.S. Cl. CPC A61K 8/894; A61K 8/064; A61K 8/31; A61K 8/37; A61Q 1/02

(57) ABSTRACT

[Problem]

The present invention has an object to provide a highly aesthetic cosmetic that is excellent in terms of its stability, finish, and spread ability at the time of application, as well as the durability of layered makeup cosmetics.

[Solution]

A water-in-oil cosmetic comprising an oil agent and the following components (A), (B) and (C), wherein the following component (C) is contained in an aqueous phase component of the water-in-oil cosmetic,
(A) one or more kinds of silicone selected from a partially crosslinked polyether-modified silicone and a partially crosslinked polyglycerin-modified silicone in an amount of 0.1 to 4% by mass in the water-in-oil cosmetic,
(B) a non-crosslinked silicone surfactant in an amount of 0.05% to 1.5% by mass in the water-in-oil cosmetic, and
(C) a silicone powder absorbing 30 parts by mass or more of squalane or ethylhexyl palmitate relative to 100 parts by mass of the silicone powder in an amount of 0.5% to 10% by mass in the water-in-oil cosmetic.

### TECHNICAL FIELD

The present invention relates to a water-in-oil cosmetic.

### BACKGROUND OF THE INVENTION

Conventionally, makeup cosmetics have been required to have durability in terms of cosmetic effects. However, after applying makeup cosmetics, there are problems such that cosmetic effects decrease over time due to sebum secreted from the skin, which causes the cosmetics to smudge or more easily adhere to other surfaces (secondary adhesion). In order to solve these problems, there are methods such that powders having oil absorbency is blended in cosmetics or makeup that absorbs sebum is applied as a makeup base.

Patent Literature 1 describes that powder having oil absorbency is blended in cosmetics in order to improve the durability of layered makeup cosmetics. However, Patent Literature 1 does not describe that powder having oil absorbency is blended into the internal aqueous phase of a water-in-oil cosmetic. When powder having oil absorbency is blended into the oil phase, there is a problem in that it absorbs the oil agent in the oil phase, which increases the viscosity of the emulsion and decreases the spread ability at the time of application.

Further, it is known that a crosslinked polyether-modified silicone is used to prepare an emulsion having a high water content and large particle sizes, resulting in a water-in-oil cosmetic with a fresh feeling when used (Patent Literatures 2, 3, and 4). In particular, the oil phases in these formulations are insufficient, so that there is a problem in that emulsification becomes difficult when powder having oil absorbency is blended into the oil phase.

### PRIOR LITERATURES

### Patent Literatures

Patent Literature 1: WO2019/176555
Patent Literature 2: Japanese Patent Application Laid-Open No. 2001-002521
Patent Literature 3: Japanese Patent Application Laid-Open No. 2011-219448
Patent Literature 4: WO2018/221174

### DISCLOSURE OF THE INVENTION

### Problems to be Solved by the Invention

The present invention has been made in consideration of the circumstances and has an object to provide a highly aesthetic cosmetic that is excellent in terms of its stability, finish, and spread ability at the time of application, as well as the durability of layered makeup cosmetics.

### Solutions to the Problems

As a result of intensive studies to achieve the aforesaid object, the present inventors found that when a silicone powder with high oil absorbency is blended into the internal aqueous phase of a water-in-oil cosmetic that includes a non-crosslinked silicone surfactant, a silicone powder with high oil absorbency, and one or more kinds of siloxane selected from a partially crosslinked polyether-modified silicone and a partially crosslinked polyglycerin-modified silicone, the resulting cosmetic has excellent stability, good spread ability, and an excellent feeling of its finish when used that does not exhibit any stickiness, is excellent in terms of the appearance of its finish and the aesthetics of the cosmetic, and demonstrates prolonged durability as it also absorbs oils such as sebum even when makeup cosmetics such as foundations are layered.

That is, the present invention provides

[1] A water-in-oil cosmetic comprising an oil agent and the following components (A), (B) and (C), wherein the following component (C) is contained in an aqueous phase component of the water-in-oil cosmetic,
(A) one or more kinds of silicone selected from a partially crosslinked polyether-modified silicone and a partially crosslinked polyglycerin-modified silicone in an amount of 0.1 to 4% by mass in the water-in-oil cosmetic,
(B) a non-crosslinked silicone surfactant in an amount of 0.05% to 1.5% by mass in the water-in-oil cosmetic, and
(C) a silicone powder absorbing 30 parts by mass or more of squalane or ethylhexyl palmitate relative to 100 parts by mass of the silicone powder in an amount of 0.5% to 10% by mass in the water-in-oil cosmetic.

[2] The water-in-oil cosmetic according to aforesaid paragraph [1], wherein an amount of the aqueous phase component comprising component (C) is 30 to 90% by mass in the water-in-oil cosmetic.

[3] The water-in-oil cosmetic according to aforesaid paragraph [1] or [2], wherein component (C) is dispersed in an internal aqueous phase of the water-in-oil cosmetic.

[4] The water-in-oil cosmetic according to any one of aforesaid paragraphs [1] to [3], wherein the silicone powder absorbs 30 parts by mass or more of squalane or ethylhexyl palmitate relative to 100 parts by mass of the silicone powder by an immersion of the silicone powder in squalane or ethylhexyl palmitate at 25° C. for 24 hours.

[5] The water-in-oil cosmetic according to any one of aforesaid paragraphs [1] to [4], wherein the silicone powder is selected from the groups consisting of silicone rubber powder coated with a silicone resin and silicone rubber particles.

[6] The water-in-oil cosmetic according to any one of aforesaid paragraphs [1] to [5], wherein the silicone powder is a silicone rubber particle of an addition reaction product of (C-1) an organohydrogen polysiloxane and (C-2) an alkenyl group containing-organopolysiloxane,
wherein one or more selected from components (C-1) and (C-2) are a silicone rubber particle having 5 to 70 mol % of alkyl groups having 6 to 30 carbon atoms bonded to the silicon atoms, based on the total mole of all substituents bonded to the silicon atoms.

[7] The water-in-oil cosmetic according to aforesaid in paragraph [6], wherein component (C-1) in the silicone rubber particle has 5 to 70 mol % of alkyl groups having 6 to 30 carbon atoms bonded to the silicon atoms, based on the total mole of all substituents bonded to the silicon atoms.

[8] The water-in-oil cosmetic according to aforesaid paragraph [6], wherein
component (C-1) is an organohydrogen polysiloxane having two or more hydrogen atoms each bonded to a silicon atom in one molecule, as represented by the following formula (1):

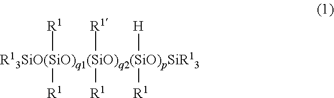

wherein R¹ is, independently of each other, a group selected from an alkyl group having 1 to 5 carbon atoms, an aryl group having 6 to 20 carbon atoms, and an aralkyl group having 7 to 20 carbon atoms, R^{1′} is an alkyl group having 6 to 30 carbon atoms, q1 and q2 are, independently of each other, an integer of 1 to 60, p is an integer of 2 to 20, and the parenthesized siloxane units, p, q1 and q2, may form a block unit or be sequenced at random, and
component (C-2) is an organopolysiloxane having two or more alkenyl groups in one molecule, as represented by the following formula (2):

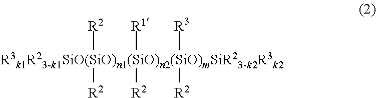

wherein R^{1′} is an alkyl group having 6 to 30 carbon atoms, R² is, independently of each other, a group selected from an alkyl group having 1 to 5 carbon atoms an aryl group having 6 to 20 carbon atoms, and an aralkyl group having 7 to 20 carbon atoms, R³ is an alkenyl group having 2 to 6 carbon atoms, n1 is an integer of 1 to 40, n2 is an integer of 0 to 50, m is an integer of 0 to 10, k1 and k2 are each an integer of 0 to 3, m+k1+k2 is an integer of 2 or more, and the parenthesized siloxane units, m, n1 and n2, may form a block unit or be sequenced at random,
wherein an amount of component (C-2) is such that a mole ratio of the alkenyl group in component (C-2) is 0.5 to 2, per mole of the hydrosilyl group in component (C-1).

[9] The water-in-oil cosmetic according to aforesaid paragraph [6], wherein the silicone rubber particle further comprises a platinum group metal catalyst (C-3) in addition to components (C-1) and (C-2).

[10] The water-in-oil cosmetic according to any one of aforesaid paragraphs [6] to [9], comprising a water dispersion of the silicone rubber particles according to any one of aforesaid paragraphs [6] to [9], wherein the water dispersion comprises
5 to 80% by mass of the silicone rubber particles,
0.01 to 15% by mass of a surfactant and
19 to 94% by mass of water.

[11] The water-in-oil cosmetic according to any one of aforesaid paragraphs [1] to [4], wherein silicone powder is spherical silicone rubber particles having a volume average particle size of 0.1 to 100 μm.

[12] The water-in-oil cosmetic according to aforesaid paragraph [5], wherein the silicone rubber particles are a vinyl dimethicone/lauryl dimethicone crosspolymer as defined in the cosmetic labeling name.

[13] The water-in-oil cosmetic according to aforesaid paragraph [5], wherein the silicone rubber powder coated with a silicone resin is at least one selected from the group consisting of vinyl dimethicone/methicone silsesquioxane crosspolymer, diphenyl dimethicone/vinyl diphenyl dimethicone/silsesquioxane crosspolymer, polysilicone-22, and polysilicone-1 crosspolymer, as defined in the cosmetic labeling name.

[14] The water-in-oil cosmetic according to aforesaid paragraph [5], wherein the silicone rubber powder coated with a silicone resin is vinyl dimethicone/methicone silsesquioxane crosspolymer as defined in the cosmetic labeling name, wherein the silicone rubber powder is coated with a silicone resin and has a hydrophilic treated surface.

[15] The water-in-oil cosmetic according to any one of aforesaid paragraphs [1] to [14], comprising (E) a volatile oil agent as the oil agent in an amount of 1 to 95% by mass in the water-in-oil cosmetic.

[16] The water-in-oil cosmetic according to any one of aforesaid paragraphs [1] to [15], comprising (F) a partially crosslinked organopolysiloxane having siloxane chain as a crosslinked segment in an amount of 0.01 to 30% by mass in the water-in-oil cosmetic.

[17] The water-in-oil cosmetic according to any one of aforesaid paragraphs [1] to [16], wherein the water-in-oil cosmetic is a water-break type.

[18] The water-in-oil cosmetic according to any one of aforesaid paragraphs [1] to [17], wherein the water-in-oil cosmetic is a foundation.

[19] A method for preparing a water-in-oil cosmetic comprising the steps of adding an aqueous phase component comprising the following components (C) and (D) into an oil phase component comprising an oil agent and the following components (A) and (B), and then stirring to obtain an emulsion,
(A) one or more kinds of silicone selected from a partially crosslinked polyether-modified silicone and a partially crosslinked polyglycerin-modified silicone in an amount of 0.1 to 4% by mass in the water-in-oil cosmetic,
(B) a non-crosslinked silicone surfactant in an amount of 0.05% to 1.5% by mass in the water-in-oil cosmetic, and
(C) a silicone powder absorbing 30 parts by mass or more of squalane or ethylhexyl palmitate relative to 100 parts by mass of the silicone powder in an amount of 0.5 to 10% by mass in the water-in-oil cosmetic,
(D) an aqueous component in an amount such that a total amount of the aqueous phase component including component (C) is 30 to 90% by mass in the water-in-oil cosmetic.

### Effects of the Invention

According to the present invention, a highly aesthetic cosmetic that is excellent in terms of its stability, finish, and spread ability at the time of application, as well as the durability of layered makeup cosmetics, can be provided.

### DETAILED DESCRIPTION OF THE INVENTION

The present invention will be described in more detail below.

In the present invention, the names of ingredients may be described under their cosmetic labeling names.

### [Component (A)]

Component (A) is one or more kinds of silicone selected from a partially crosslinked polyether-modified silicone and a partially crosslinked polyglycerin-modified silicone. Each of them is a partially crosslinked modified silicone having polyether chains or polyglycerin chains, respectively, as crosslinked segments. Component (A) is blended into the cosmetic, so that the cosmetic can have excellent stability and be emulsified even with a high internal aqueous phase.

The partially crosslinked polyether-modified silicone is not particularly limited and may be a three-dimensionally crosslinked product obtained by crosslinking organopolysiloxane chains with polyether. The partially crosslinked polyether-modified silicone may be a known compound blended into cosmetics and is known under the cosmetic labeling names such as dimethicone/PEG-10/15 crosspolymer. The partially crosslinked polyether-modified silicone is commercially available as a swelling product that includes silicone oil or another oil, and is commercially available under product names such as KSG-210, KSG-240, and KSG-270 (all of them are manufactured by Shin-Etsu Chemical Co., Ltd.). Examples of a crosslinked product having an alkyl branch in its main chain (alkyl-modified, partially crosslinked polyether-modified silicone) include PEG-15/lauryl dimethicone crosspolymer and PEG-15/lauryl polydimethylsiloxyethyl dimethicone crosspolymer. The crosslinked product having an alkyl branch in its main chain is commercially available as a swelling product that includes silicone oil or another oil, and examples thereof include KSG-310, KSG-310Z, KSG-330, KSG-340, KSG-320Z, KSG-350Z, and KSG-360Z (all of them are manufactured by Shin-Etsu Chemical Co., Ltd.).

The partially crosslinked polyglycerin-modified silicone is not particularly limited and may be a three-dimensionally crosslinked product obtained by crosslinking organopolysiloxane chains with polyglycerin. The partially crosslinked polyglycerin-modified silicone may be a known compound blended into cosmetics and is known under the cosmetic labeling names such as dimethicone/polyglycerin-3 crosspolymer. The partially crosslinked polyglycerin-modified silicone is commercially available as a swelling product that includes silicone oil or another oil, and is commercially available under product names such as KSG-710 (manufactured by Shin-Etsu Chemical Co., Ltd.). Examples of a crosslinked product having an alkyl branch in its main chain include lauryl dimethicone/polyglycerin-3 crosspolymer and polyglyceryl-3/lauryl polydimethylsiloxyethyl dimethicone crosspolymer. The crosslinked product having an alkyl branch in its main chain is commercially available as a swelling product that includes silicone oil or another oil, and is commercially available under product names such as KSG-810, KSG-820, KSG-820Z, KSG-830, KSG-840, and KSG-850Z (all of them are manufactured by Shin-Etsu Chemical Co., Ltd.).

The amount of component (A) is 0.1% to 4% by mass or preferably 0.2% to 2% by mass, based on the total amount of the cosmetic. If the amount is less than 0.1% by mass, that may cause the emulsion stability of the cosmetic to decrease. If the amount is more than 4% by mass, that may cause the freshness of the cosmetic to be lost and the feeling when used at the time of application to deteriorate.

Component (A) may be used alone or in an appropriate combination of two or more kinds. For example, in a combination of a partially crosslinked polyether-modified silicone and a partially crosslinked polyglycerin-modified silicone, when the relative blending ratio of the partially crosslinked polyether-modified silicone is high, the spread ability of the cosmetic tends to improve, and when the relative blending ratio of the partially crosslinked polyglycerin-modified silicone is high, the cosmetic tends to have a moist and gentle feeling when used. The blending ratios are appropriately determined and, thereby, the feeling when used may be controlled.

### [Component (B)]

Component (B) is a non-crosslinked silicone surfactant. Component (B) is blended in the cosmetics, so that the storage stability of the cosmetic and its feeling when used due to the adjustment of emulsified particle sizes may be adjusted. Component (B) is not particularly limited and may be a non-crosslinked silicone surfactant that is blended into conventional cosmetics, and may be used alone or in an appropriate combination of two or more kinds. Among such surfactants, straight-chain or branched polyoxyethylene-modified organopolysiloxane, straight-chain or branched polyoxyethylene-polyoxypropylene-modified organopolysiloxane, straight-chain or branched polyglycerin-modified organopolysiloxane, straight-chain or branched polyoxyethylene- and alkyl-co-modified organopolysiloxane, straight-chain or branched polyoxyethylene-polyoxypropylene- and alkyl-co-modified organopolysiloxane, silicone acrylate graft copolymers, and the like are preferable. Specific examples thereof include polyether-modified silicones such as KF-6011, KF-6013, KF-6043, KF-6017, and KF-6017P; silicone-branched polyether-modified silicones such as KF-6028 and KF-6028P; silicone-branched polyglycerin-modified silicones such as KF-6100, KF-6104, and KF-6106; silicone acrylate graft copolymers such as KP-578; silicone- and alkyl-branched polyether-modified silicones such as KF-6038; alkyl-branched polyether-modified silicones such as KF-6048; and silicone- and alkyl-branched polyglycerin-modified silicones such as KF-6105 and KF-6115 (all of them are manufactured by Shin-Etsu Chemical Co., Ltd.).

The amount of component (B) is 0.05% to 1.5% by mass, preferably 0.1% to 1.0% by mass, or further preferably 0.2% to 0.8% by mass, based on the total amount of the present cosmetic. When the amount is 0.05% by mass or more, the emulsion stability of the cosmetic further improves. When the amount is 1.5% by mass or less, that results in more freshness and an excellent feeling when used at the time of application.

### [Component (C)]

Component (C) is a silicone powder with high oil absorbency. The silicone powder may be used alone or in an appropriate combination of two or more kinds. Component (C) is blended in the cosmetics and, thereby, the durability of makeup improves due to the absorption of sebum or excess oil, and a finish that is excellent in terms of appearance and feeling is obtained.

The silicone powder with high oil absorbency absorbs 30 parts by mass or more, preferably 60 parts by mass or more, or further preferably 80 parts by mass or more, relative to 100 parts by mass of component (C), of at least one kind of hydrocarbon oil or ester oil. For hydrocarbon oils and ester oils, which are sebum-like components, an amount of absorption less than 30 parts by mass may cause sufficient transfer resistance and durability to be unobtained and the feeling to become heavy. The upper limit of the amount of absorption is not particularly limited, and the amount of absorption may be, for example, 500 parts by mass or less, or particularly 300 parts by mass or less, in terms of practicality. In particular, in the present invention, a product that absorbs 30 parts by mass or more, relative to 100 parts by mass of the silicone powder, of squalane or ethylhexyl palmitate is considered to be a silicone powder having “high oil absorbency.”

In the present invention, the silicone powder preferably consists of a silicone rubber or a silicone elastomer which has high oil absorbency as described above. That is, in the present invention, component (C) is preferably powder consisting of a silicone rubber or a silicone elastomer. Component (C) is preferably powder consisting of the silicone rubber or the silicone elastomer that absorbs 30 parts by mass or more, preferably 60 parts by mass or more, further preferably 80 parts by mass or more of at least one kind of oily substance selected from a group consisting of hydrocarbon oils or ester oils, particularly squalane or ethylhexyl palmitate. The amount is relative to 100 parts by mass of the silicone rubber or the silicone elastomer. The upper limit of the amount of absorption is as described above.

Preferred is that the amount of oil absorption is the amount of an oily substance absorbed in the silicone rubber when the silicone rubber is immersed in the aforesaid oily substance at 25° C. for 24 hours, which is shown as a value relative to 100 g of the silicone rubber. More specifically, the amount of an oily substance absorbed in a sheet with a thickness of 1 mm when the sheet is immersed in the oily substance at 25° C. for 24 hours is converted to a value relative to 100 g of the silicone rubber, in particular, the sheet has 30 mm×30 mm square and a thickness of 1 mm and is formed from the silicone rubber cured product constituting the silicone powder. This amount is shown as the amount of the oil absorption.

Furthermore, for silicone powders whose amounts of oil absorption cannot be determined according to the aforementioned method, the amount of oil absorption is to be determined according to the method explained in below.

The silicone powder and the oily substance described above are placed in a glass bottle and shaken for 15 minutes, then left to stand at 25° C. for 24 hours. The solid content is then separated with a pressure filter, and the mass of the solid content is measured. The amount of oil absorption may be determined by subtracting the mass of the silicone powder before the oil absorption from the mass of the solid content and converting the resulting value to a value relative to 100 g of the silicone powder.

Hydrocarbon oils and ester oils, which are oily substances, are used as cosmetic ingredients. Examples of the hydrocarbon oils include straight-chain or branched hydrocarbon oils, and they may be volatile hydrocarbon oils or non-volatile hydrocarbon oils. Examples of the hydrocarbon oils include squalanes such as synthesized squalane and vegetable squalane; squalene; light liquid isoparaffin; liquid paraffin; liquid isoparaffin; and hydrogenated polyisobutene. Examples of the ester oils include diethylhexyl succinate, diethylhexyl sebacate, dibutyloctyl sebacate, diisocetyl adipate, diisostearyl malate, di-2-heptylundecyl adipate, propylene glycol dicaprate, neopentyl glycol dicaprate, trimethylolpropane triethylhexanoate, pentaerythrityl tetraethylhexanoate, ethyl linoleate, isopropyl isostearate, isopropyl linoleate, butyl stearate, octyl palmitate, ethylhexyl palmitate, ethylhexyl stearate, decyl oleate, myristyl myristate, cetyl ethylhexanoate, cetyl octanoate, cetyl palmitate, isocetyl dimethyloctanoate, isocetyl myristate, isocetyl palmitate, isocetyl stearate, isocetyl isostearate, isostearyl palmitate, octyldodecyl neopentanoate, octyldodecyl oleate, and octyldodecyl myristate.

The silicone powder is preferably a spherical powder, in view of the feeling when used. In this specification, “spherical” means that the shape of the fine particles may be a perfect sphere or a deformed sphere. More specifically, the longest axis length/the shortest axis length (the aspect ratio), on average, may be within the conventional range of 1 to 4 and is preferably 1 to 2, more preferably 1 to 1.6, or even more preferably 1 to 1.4. The shape of the fine particles may be confirmed by observing the fine particles with an optical microscope, an electron microscope, or the like.

The average particle size of the silicone powder is not particularly limited. When the silicone powder is blended into cosmetics, the volume average particle size (cumulative D50, also known as the median diameter) determined with a method according to a Coulter counter is preferably 0.1 to 100 μm, further preferably 1 to 40 μm, in view of the silky feeling and smoothness.

The amount of the silicone powder in the cosmetic is 0.5% to 10% by mass, preferably 0.5% to 8% by mass, more preferably 1.8% to 6% by mass, or even more preferably 3.0% to 5% by mass. If the amount is less than 0.5% by mass, that may cause a sufficient oil absorption effect to be unobtained. If the amount is more than 10% by mass, that may cause the silicone powder to not be fully contained in the internal aqueous phase, resulting in the breakdown of the emulsion.

Preferable examples of the silicone powder with high oil absorbency include silicone rubber powder coated with silicone resin and silicone rubber particles (crosslinked silicone powder).

Examples of the silicone rubber powder coated with silicone resin and having high oil absorbency include a silicone rubber powder consisting of 100 parts by mass of silicone rubber spherical fine particles having a volume average particle size of 0.1 to 100 μm and 0.5 to 25 parts by mass of polyorganosilsesquioxane coated on the surfaces of the silicone rubber.

Examples of the silicone rubber powder coated with silicone resin include vinyl dimethicone/methicone silsesquioxane crosspolymer, diphenyl dimethicone/vinyl diphenyl dimethicone/silsesquioxane crosspolymer, polysilicone-22, and polysilicone-1 crosspolymer, as defined by the cosmetic labeling name. They are commercially available under product names such as KSP-100, KSP-101, KSP-102, KSP-105, KSP-300, KSP-411, and KSP-441 (all of them are manufactured by Shin-Etsu Chemical Co., Ltd.). Further, examples of the silicone rubber powder coated with silicone resin and having surfaces subjected to a hydrophilic treatment include KSP-100W (manufactured by Shin-Etsu Chemical Co., Ltd.). Among them, vinyl dimethicone/methicone silsesquioxane crosspolymer (KSP-100, KSP-101, and KSP-102) and polysilicone-1 crosspolymer (KSP-411), which have high amounts of silicone oil absorption, diphenyl dimethicone/vinyl diphenyl dimethicone/silsesquioxane crosspolymer (KSP-300), which has high oil absorbency for UV absorbers, and polysilicone-22 (KSP-441), which has high oil absorption efficiency for a wide variety of oil agents, are exemplified. Among them, polysilicone-22 (KSP-441), which is highly effective in terms of the durability of makeup, is preferable. The silicone rubber powders coated with silicone resin are preferable in view of feeling improvement effects such as the prevention of stickiness and to contouring effects for wrinkles, pores, and the like.

The silicone rubber particles are known under names such as dimethicone/vinyl dimethicone crosspolymer and vinyl dimethicone/lauryl dimethicone crosspolymer. They may be commercially available as: powders; or swelling products or water dispersions that include silicone oil. For example, they are commercially available under product names such as KMP-598 and KSG-016F (all of them are manufactured by Shin-Etsu Chemical Co., Ltd.). These powders may be used alone or in combination of two or more kinds. Silicone rubber particles are preferable in view of feeling improvement effects such as the prevention of stickiness and to contouring effects for wrinkles, pores, and the like.

### [Silicone Rubber Particles]

Silicone rubber particles that are suitable as component (C) are spherical silicone rubber particles that are products of an addition reaction with an organohydrogen polysiloxane (C-1) and an alkenyl-group-containing organopolysiloxane (C-2), and one or more selected from components (C-1) and (C-2) have an alkyl group having 6 to 30 carbon atoms bonded to a silicon atom in an amount of 5 mol % to 70 mol %, based on the total number of moles of all substituents bonded to silicon atoms. Each component is described in detail below.

### [(C-1) Organohydrogen Polysiloxane]

The organohydrogen polysiloxane (C-1) is preferably an organohydrogen polysiloxane having two or more hydrogen atoms each bonded to a silicon atom in one molecule, as represented by the following formula (1):

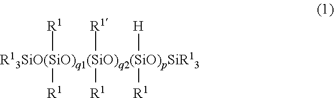

R¹ is, independently of each other, a group selected from an alkyl group having 1 to 5 carbon atoms, preferably 1 to 4 carbon atoms, or further preferably 1 to 3 carbon atoms, an aryl group having 6 to 20 carbon atoms, preferably 6 to 16 carbon atoms, or further preferably 6 to 12 carbon atoms, and an aralkyl group having 7 to 20 carbon atoms, preferably 7 to 16 carbon atoms, or further preferably 7 to 12 carbon atoms.

Examples of the alkyl group include a methyl group, an ethyl group, a propyl group, a butyl group, a pentyl group, and a cyclopentyl group. Examples of the aryl group include a phenyl group, a tolyl group, and a naphthyl group. Examples of the aralkyl group include a benzyl group, a phenethyl group, and a 2-phenylpropyl group. Among them, an alkyl group having 1 to 5 carbon atoms is preferable, and a methyl group is more preferable.

Furthermore, R^{1′} is an alkyl group having 6 to 30 carbon atoms, preferably 8 to 20 carbon atoms, or further preferably 10 to 15 carbon atoms. Examples thereof include alkyl groups such as a hexyl group, a heptyl group, an octyl group, a decyl group, an undecyl group, a dodecyl group, a tetradecyl group, a pentadecyl group, a hexadecyl group, a heptadecyl group, an octadecyl group, a nonadecyl group, an icosyl group, a henicosyl group, a docosyl group, a tricosyl group, a tetracosyl group, and a triacontyl group; and cycloalkyl groups such as a cyclohexyl group and a cycloheptyl group.

q1 is an integer of 1 to 60 or preferably an integer of 10 to 50. q2 is an integer of 1 to 60, preferably an integer of 1 to 50, or further preferably an integer of 5 to 30.

p is an integer of 2 to 20 or preferably an integer of 3 to 10.

In formula (1), the parenthesized siloxane units, p, q1, and q2, may form a block unit or be sequenced at random.

Component (C-1) may be used alone or in combination of two or more kinds.

The kinematic viscosity of component (C-1) is preferably 100,000 mm²/s or less, or further preferably 10,000 mm²/s or less. When the kinematic viscosity is 100,000 mm²/s or less, that makes it easier to obtain silicone rubber particles with a narrow particle size distribution. The lower limit of the kinematic viscosity is not particularly limited, and in terms of practicality, the kinematic viscosity may be, for example, 0.4 mm²/s or more, particularly 2 mm²/s or more. Note that, in the present specification, the kinematic viscosity is a value determined with a Cannon-Fenske viscometer at 25° C. described in JIS Z 8803:2011.

### [(C-2) Alkenyl Group Containing-Organopolysiloxane]

The alkenyl group containing-organopolysiloxane (C-2) is preferably an organopolysiloxane having two or more alkenyl groups in one molecule, as represented by the following formula (2).

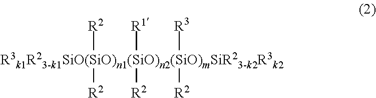

R^{1′} has the same definition as the R^{1′} described in formula (1) for component (C-1), and preferable ranges are exemplified by those described for R^{1′} in formula (1).

Examples of R² are the same substituents as the definition of R¹ described in formula (1) for component (C-1), and preferable ranges are exemplified by those described for R¹.

R³ is an alkenyl group having 2 to 6 carbon atoms or preferably 2 or 3 carbon atoms. Examples of the alkenyl group include a vinyl group, an allyl group, a butenyl group, a pentenyl group, and a hexenyl group. A vinyl group or an allyl group is preferable, and a vinyl group is more preferable.

n1 is an integer of 1 to 40 or preferably an integer of 5 to 30. n2 is an integer of 0 to 50 or preferably 0 or an integer of 1 to 20.

m is an integer of 0 to 10 or preferably 0 or an integer of 1 to 5. k1 and k2 are each an integer of 0 to 3, or preferably k1 and k2 are both 1, and m+k1+k2 is an integer of 2 or more.

The parenthesized siloxane units, m, n1, and n2, may form a block unit or be sequenced at random.

Component (C-2) may be used alone or in combination of two or more kinds.

The kinematic viscosity of component (C-2) is preferably 100,000 mm²/s or less, or further preferably 10,000 mm²/s or less. When the kinematic viscosity is 100,000 mm²/s or less, that makes it easier to obtain silicone fine particles with a narrow particle size distribution. The lower limit of the kinematic viscosity is not particularly limited, and in terms of practicality, the kinematic viscosity may be, for example, 0.7 mm²/s or more, or particularly 2 mm²/s or more.

Furthermore, preferred is that in one or more selected from components (C-1) and (C-2), that is, in component (C-1) or component (C-2), or in both, 5 mol % to 70 mol %, preferably 8 mol % to 60 mol % of the total number of all substituents bonded to the silicon atoms are an alkyl group having 6 to 30 carbon atoms, preferably 8 to 20 carbon atoms, further preferably 10 to 15 carbon atoms. More preferably, only component (C-1) satisfies the aforementioned conditions. Note that the aforesaid number of the substituents bonded to the silicon atoms does not include the number of the hydrogen atoms bonded to the silicon atoms.

If the proportion of the alkyl group is less than the lower limit, that may cause the aforesaid amount of absorption of the oily substance to more easily decrease. If the proportion of the alkyl group is more than the aforesaid upper limit, the reactivity between the organohydrogen polysiloxane of component (C-1) and the organopolysiloxane having an olefin hydrocarbon group of component (C-2) may tend to be less and the rubber hardness of the resulting cured product may tend to be low.

When the aforesaid conditions are satisfied, in formula (1) for component (C-1), q2 is such an integer that the number of moles of R^{1′} is 5 mol % to 70 mol %, preferably 8 mol % to 60 mol %, based on the total number of moles of all substituents (R¹ and R^{1′}) bonded to the silicon atoms.

Similarly, in formula (2) for component (C-2), n2 is such an integer that the number of moles of R^{1′} is 5 mol % to 70 mol %, preferably 8 mol % to 60 mol %, based on the total number of moles of all substituents (R² and R^{1′}) bonded to the silicon atoms. Note that, preferably, the number of the alkenyl group bonded to the silicon atom represented by R³ is not included in the number of the substituent bonded to the silicon atom.

Furthermore, in view of reactivity, substituents other than the monovalent aliphatic saturated hydrocarbon group (R^{1′}) having 6 to 30 carbon atoms in components (C-1) and (C-2) are preferably a methyl group.

The amount of component (C-2) is such that a mole ratio of the alkenyl group in component (C-2) is 0.5 to 2, per mole of the hydrosilyl group in component (C-1).

The number of hydrosilyl groups in component (C-1) and the number of alkenyl groups in component (C-2) are not two at the same time. That is, when component (C-1) has two hydrosilyl groups, one or more kinds of component (C-2) will be organosiloxanes having three or more alkenyl groups. Alternatively, when component (C-2) has two alkenyl groups, one or more kinds of component (C-1) will be organohydrogen siloxanes having three or more hydrosilyl groups.

The present component (C) is preferably a cured product of an addition reaction of a silicone composition including a platinum group metal catalyst (C-3) in addition to the organohydrogen polysiloxane (C-1) and the alkenyl-group-containing organopolysiloxane (C-2). Component (C-3) is described in detail below.

### [Platinum Group Metal Catalyst (C-3)]

The platinum group metal catalyst (C-3) is a catalyst for promoting the addition reaction between a hydrosilyl group in component (C-1) and an alkenyl group in component (C-2). Component (C-3) may be used alone or in combination of two or more kinds.

As component (C-3), well-known catalysts used in hydrosilylation reactions are exemplified. Examples thereof include platinum group metal simple substances such as platinum (including platinum black), rhodium, and palladium; platinum chlorides, chloroplatinic acids, and chloroplatinates such as H₂PtCl₄·kH₂O, H₂PtCl₆·kH₂O, NaHPtCl₆·kH₂O, KHPtCl₆·kH₂O, Na₂PtCl₆·kH₂O, K₂PtCl₄·kH₂O, PtCl₄·kH₂O, PtCl₂, and Na₂HPtCl₄·kH₂O (provided that, in the formulas, k is an integer of 0 to 6 or preferably 0 or 6); alcohol-modified chloroplatinic acids (see U.S. Pat. No. 3,220,972 B); olefin complexes of platinum chloride and chloroplatinic acid (see U.S. Pat. Nos. 3,159,601 B, 3,159,662 B, and 3,775,452 B) and vinyl-group-containing siloxane complexes of chloroplatinic acid and platinum; products obtained by supporting a platinum group metal such as platinum black or palladium with a carrier such as alumina, silica, carbon, or the like; rhodium-olefin complexes; and chlorotris(triphenylphosphine) rhodium (Wilkinson's catalyst).

The amount of component (C-3) may be an effective amount as a hydrosilylation reaction catalyst. The amount of component (C-3) is such that the amount of the platinum group metal in component (C-3) is generally about 0.1 to 500 ppm, preferably about 0.5 to 200 ppm, or further preferably about 1 to 100 ppm, in terms of mass, relative to the total amount of the composition.

The silicone rubber constituting the silicone rubber particles preferably does not exhibit any stickiness, and its rubber hardness value, as determined by an ASKER Durometer Type C specified in the Society of Rubber Industry, Japan Standard (SRIS), 0101, is preferably in the range of 10 to 95, or further preferably in the range of 20 to 85. When the rubber hardness value is in the range of 10 to 95, that makes it easier to achieve a smooth and gentle feeling throughout.

Furthermore, the silicone rubber particles may contain silicone oils, organosilanes, inorganic powders, organic powders, antioxidants, and the like.

### [Water-Based Dispersion of the Silicone Rubber Particles]

The silicone rubber particles (C) are mixed as a water-based dispersion or preferably as a water dispersion, in which the silicone rubber particles (C) are dispersed in advance in the aqueous component (D) described below, which is preferable in view of the ease of cosmetic preparation. The silicone rubber particles (C) may be subjected to a hydrophilic treatment and then mixed with the aqueous component (D), or the silicone rubber particles (C) may be mixed with a hydrophilic surfactant and then mixed with the aqueous component (D). A water-based dispersion obtained by emulsion polymerization in a mixture including the aqueous component (D) is preferable in view of the reduction of surfactants and to the stability of the aqueous phase. In particular, when a water dispersion of silicone rubber particles is used, a cosmetic with high viscosity stability is obtained. Preferably, the water dispersion includes the silicone rubber particles at an amount of 5% to 80% by mass, surfactants at an amount of 0.01% to 15% by mass, and water at an amount of 19% to 94% by mass, based on the total mass of the water dispersion.

The amount of the silicone rubber particles is 5% to 80% by mass, preferably 20% to 70% by mass, or further preferably 40% to 65% by mass, based on the total mass of the water dispersion of the silicone rubber particles. If the amount of the silicone rubber particles is less than the lower limit, that may cause the productivity per silicone rubber particle to be low and the amount of the water dispersion added to the cosmetic to increase, which is inefficient and undesirable. If the amount of the silicone rubber particles is more than the upper limit, that may cause the viscosity of the water dispersion to increase and handling to become difficult.

### Surfactant

The surfactant functions as a dispersant for the silicone rubber particles in the water-based dispersion or the cosmetic. As described below, it also functions as an emulsifier for liquid silicone in the production of the water dispersion of the silicone rubber particles.

The surfactant is not particularly limited, and may be a nonionic surfactant, an anionic surfactant, a cationic surfactant, or an amphoteric surfactant. The surfactant may be used alone or in an appropriate combination of two or more kinds.

Examples of the nonionic surfactant include polyoxyethylene alkyl ethers, polyoxyethylene polyoxypropylene alkyl ethers, polyoxyethylene alkyl phenyl ethers, polyethylene glycol fatty acid esters, sorbitan fatty acid esters, polyoxyethylene sorbitan fatty acid esters, polyoxyethylene sorbitol fatty acid esters, glycerin fatty acid esters, polyoxyethylene glycerin fatty acid esters, polyglycerin fatty acid esters, propylene glycol fatty acid esters, polyoxyethylene castor oil, polyoxyethylene hydrogenated castor oil, polyoxyethylene hydrogenated castor oil fatty acid esters, polyoxyethylene alkyl amines, and polyoxyethylene fatty acid amides.

In addition, the partially crosslinked polyether-modified silicone and the partially crosslinked polyglycerin-modified silicone corresponding to component (A) are not used as this surfactant, but the non-crosslinked silicone surfactant corresponding to component (B) may be used as this surfactant. Examples of the non-crosslinked silicone surfactant include polyoxyethylene-modified organopolysiloxane and polyoxyethylene-polyoxypropylene-modified organopolysiloxane. When the non-crosslinked silicone surfactant is used as a component contained in the water-based dispersion, the non-crosslinked silicone surfactant included in the water-based dispersion is not included in the amount of component (B) described.

Examples of the anionic surfactant include alkyl sulfates such as sodium lauryl sulfate, polyoxyethylene alkyl ether sulfates, polyoxyethylene alkyl phenyl ether sulfates, fatty acid alkylol amide sulfates, alkyl benzene sulfonates, polyoxyethylene alkyl phenyl ether sulfonates, α-olefin sulfonates, α-sulfofatty acid esters and salts, alkyl naphthalene sulfonic acids, alkyl diphenyl ether disulfonates, alkane sulfonates, N-acyl taurates, dialkyl sulfosuccinates, monoalkyl sulfosuccinates, polyoxyethylene alkyl ether sulfosuccinates, fatty acid salts, polyoxyethylene alkyl ether carboxylates, N-acyl amino acid salts, monoalkyl phosphates, dialkyl phosphates, and polyoxyethylene alkyl ether phosphates.

Examples of the cationic surfactant include alkyltrimethylammonium salts, dialkyldimethylammonium salts, polyoxyethylene alkyldimethylammonium salts, dipolyoxyethylene alkylmethylammonium salts, tripolyoxyethylene alkylammonium salts, alkylbenzyldimethylammonium salts, alkylpyridinium salts, monoalkyl amine salts, and monoalkyl amidoamine salts.

Examples of the amphoteric surfactant include alkyl dimethyl amine oxide, alkyl dimethyl carboxybetaine, alkyl amidopropyl dimethyl carboxybetaine, alkyl hydroxysulfobetaine, and alkyl carboxymethyl hydroxyethyl imidazolinium betaine.

The amount of the surfactant is 0.01% to 15% by mass, preferably 0.1% to 10% by mass, or further preferably 0.5% to 5% by mass, based on the total mass of the water dispersion. If the amount is less than the aforesaid lower limit, that may cause the emulsification of liquid silicone during the production stage to become impossible, making it difficult to obtain particles with a narrow particle size distribution. There is also a risk that the stability of the water dispersion will decrease. Furthermore, if the amount is more than the aforesaid upper limit, that is not desirable because the water dispersion efficiency of the silicone rubber particles is not increased, and there is a risk of increasing irritation to the skin.

Water is a dispersion medium for the silicone rubber particles. The amount of water is 19% to 94% by mass or preferably 30% to 80% by mass, based on the total mass of the water dispersion. Water is included in the aqueous component (D) described below.

### Production Method of the Water Dispersion of the Silicone Rubber Particles

The water dispersion of the silicone rubber particles may be produced according to known methods. Example of the methods incudes a method in which a surfactant and water are added to a mixed dissolved product of the organohydrogen polysiloxane (C-1) and the olefin-hydrocarbon-group-having organopolysiloxane (C-2), emulsification is performed, the platinum group metal catalyst (C-3) is added thereto after emulsification and, then, an addition reaction is performed. This method is known as emulsion polymerization.

For emulsification, a general emulsifying and dispersing machine may be used. Examples thereof include high-speed rotating centrifugal radial stirrers such as a homodisper; high-speed rotating shear stirrers such as a homomixer; high-pressure-jet emulsifying and dispersing machines such as a homogenizer; colloid mills; and ultrasonic emulsifiers.

When the platinum group metal catalyst (C-3) has poor dispersibility in water, the platinum group metal catalyst (C-3) is preferably added to the emulsion with the platinum group metal catalyst (C-3) dissolved in a surfactant. The addition reaction may be performed at room temperature (1° C. to 30° C.), but if the reaction is not completed, it may be performed under heating at less than 100° C.

### [Component (D)]

Component (D) is an aqueous component that may be blended into conventional cosmetics, but not particularly limited. More specifically, examples thereof include water, a moisturizer, a water-soluble polymer compound, a skin-beautifying component, a water-soluble inorganic salt, and a pH adjuster. These may be used alone or in an appropriate combination of two or more kinds.

Examples of water include sea water, onsen-sui (hot spring water), and peat water, as defined by the cosmetic labeling name, in addition to purified water and distilled water from fruits and plants generally used in cosmetics.

Examples of the moisturizer include lower alcohols such as alcohol and isopropyl alcohol; sugar alcohols such as sorbitol, maltose, and xylitol; polyhydric alcohols such as butylene glycol, dibutylene glycol, propylene glycol, dibutylene glycol, pentylene glycol, decanediol, octanediol, hexanediol, erythritol, glycerin, diglycerin, and polyethylene glycol; glucose; glyceryl glucoside; betaine; chondroitin sulfate; pyrrolidone carboxylic acid; polyoxyethylene methyl glucoside; and polyoxypropylene methyl glucoside. The amount of the moisturizer in the aqueous component is preferably 5% to 70% by mass, more preferably 8% to 50% by mass, or even more preferably 10% to 30% by mass. On account of the moisturizer, the stability of the cosmetic is improved and water-break properties are adjusted. Among them, in particular, moisturizers having two or more hydroxyl groups are preferable. More specifically, examples thereof include butylene glycol, glycerin, and sorbitol. The amount of alcohol having two or more hydroxyl groups in the moisturizer is preferably 30% to 100% by mass, more preferably 50% to 100% by mass, or even more preferably 65% to 100% by mass.

Examples of the water-soluble polymer compound include natural water-soluble polymer compounds such as carrageenan, hyaluronate, and xanthan gum; semi-synthetic water-soluble polymer compounds such as hydroxyethylcellulose, hydroxypropyl methylcellulose, and carboxymethyl cellulose; synthetic water-soluble polymer compounds such as polyvinyl alcohol, polyvinylpyrrolidone, and carboxyvinyl polymers; and inorganic water-soluble polymer compounds such as bentonite and Laponite. The amount of the water-soluble polymer compound in the aqueous component is preferably 0.01% to 1% by mass. On account of the water-soluble polymer compound, the stability of the cosmetic is improved and water-break properties are adjusted.

Examples of the skin-beautifying component include skin-lightening agents such as arbutin, as well as ascorbic acid and its derivatives; anti-inflammatory agents such as allantoin and glycyrrhizinate; and blood circulation promoters such as benzyl nicotinate. The amount of the skin-beautifying component may be appropriately adjusted according to conventional cosmetics, as long as the effects of the present invention are not impaired.

Examples of other components include water-soluble inorganic salts and pH adjusters such as sodium chloride, magnesium sulfate, sodium hydroxide, potassium hydroxide, arginine, citric acid, sodium citrate, lactic acid, and glycolic acid. The amount of the other components may be appropriately adjusted according to conventional cosmetics, as long as the effects of the present invention are not impaired.

The total amount of the aqueous component is 30% to 90% by mass, preferably 50% to 90% by mass, or further preferably 60% to 85% by mass, based on the entire cosmetic of the present invention. In the present water-in-oil cosmetic, the silicone rubber particles (C) described above are dispersed in the internal aqueous phase. The amount of aqueous phase components including component (C) is 30% to 90% by mass, preferably 50% to 90% by mass, or further preferably 60% to 85% by mass in the cosmetic.

The amount of the aqueous component is increased and each component is blended with the amounts described above and, thereby, a cosmetic having an emulsion with large particle sizes are obtained. When the cosmetic is applied, the water-in-oil emulsion breaks due to the shear force of the application, and the internal aqueous phase bursts out as water droplets. The cosmetic (water-break cosmetic) is a water-in-oil cosmetic with a fresh feeling.

Note that the amount of the aqueous component, preferably water, included in the water-based dispersion of the silicone powder (C) described above is included in the total amount of the aqueous component (D) described above.

In the water-break cosmetic having an insufficient amount of the oil phase, when the silicone rubber powder (C) is blended into the oil phase, emulsification becomes difficult and the long-term stability of the cosmetic decreases. On the other hand, the present invention provides a cosmetic in which the silicone rubber powder (C) is dispersed in the internal aqueous phase, and provides a stable water-break cosmetic.

### [Component (E)]

Component (E) is a volatile oil agent and may be used alone or in an appropriate combination of two or more kinds. On account of component (E) blended, the transfer resistance of the cosmetic improves. On account of the volatile oil agent (E) and the silicone rubber powder (C) blended together, it is believed that, after applying the cosmetic, component (C) absorbs sebum and excess oil after component (E) volatilizes. In the present invention, the volatile oil agent may refer to an oil agent having a boiling point of 250° C. or less. The amount of the volatile oil agent in the cosmetic is 1% to 95% by mass, preferably 5% to 65% by mass, or further preferably 5% to 30% by mass.

More specifically, examples thereof include oily volatile components such as dimethylpolysiloxane (KF-96L-1cs, KF-96L-1.5cs, KF-96L-2cs, and the like, manufactured by Shin-Etsu Chemical Co., Ltd.), octamethylcyclotetrasiloxane (D4), decamethylcyclopentasiloxane (KF-995 (D5), manufactured by Shin-Etsu Chemical Co., Ltd.), dodecamethylcyclohexasiloxane (D6), tris(trimethylsilyloxy)methylsilane (TMF-1.5, manufactured by Shin-Etsu Chemical Co., Ltd.), ethyl methicone (KF-4422, manufactured by Shin-Etsu Chemical Co., Ltd.), light isoparaffin, undecane, and isododecane, which have a boiling point of 250° C. or less.

### [Component (F)]

Component (F) is a partially crosslinked organopolysiloxane other than component (A) and is preferably blended into the cosmetic in view of the improvement of the storage stability of the preparation and the feeling when used. Component (F) may be used alone or in an appropriate combination of two or more kinds. Unlike component (A), component (F) is a compound that does not have a polyether or polyglycerin structure in its molecular structure and is an elastomer that has structural viscosity by swelling an oil agent. In addition, it is different from component (C), which is powder. Examples of component (F) include dimethicone/vinyl dimethicone crosspolymer, dimethicone/phenyl vinyl dimethicone crosspolymer, vinyl dimethicone/lauryl dimethicone crosspolymer, and lauryl polydimethylsiloxyethyl dimethicone/bis-vinyl dimethicone crosspolymer. They are commercially available as swelling products that include oils which are liquid at room temperature. Specific examples thereof include KSG-15, KSG-1510, KSG-16, KSG-1610, KSG-18A, KSG-19, KSG-41A, KSG-42A, KSG-43, KSG-44, KSG-042Z, KSG-045Z, and KSG-048Z, manufactured by Shin-Etsu Chemical Co., Ltd. When component (F) is blended, the amount of component (F) in the cosmetic is preferably 0.01% to 30% by mass, further preferably 0.1% to 4% by mass.

### [Cosmetic]

The present invention may be applicable to various cosmetics, and may be preferably applicable to cosmetics applied externally to the skin, such as skin care cosmetics, makeup cosmetics, antiperspirant cosmetics, and UV protection cosmetics; cosmetics applied externally to hair, such as hair cosmetics; and nail cosmetics. Examples of the skin care cosmetics include skin lotions, milky lotions, creams, cleansing products, facial masks, skin care oils and liquids, products for massages, serums, emollient oils, cleansers, deodorants, hand creams, lip balms, and wrinkle concealers. Examples of the makeup cosmetics include makeup bases, foundations, concealers, face powders, blushes, colored eyeshadows, eyeshadows, mascaras, eyeliners, eyebrow products, and lipsticks. Examples of the antiperspirant cosmetics include roll-on antiperspirants, antiperspirant creams, antiperspirant solutions, and antiperspirant sticks. Examples of the UV protection cosmetics include sunscreen oils, sunscreen milky lotions, and sunscreen creams. Examples of the hair cosmetics include shampoos, conditioners, treatments, and styling products.

Among them, the present cosmetic is most suitably used as a makeup base cosmetic because it demonstrates prolonged durability by absorbing oils such as sebum even when makeup cosmetics such as foundations are layered.

The form of the present cosmetic is not particularly limited and may be a water-in-oil cosmetic. The form of the emulsion may be either a W/O emulsion or an O/W/O emulsion. The definition of the water-in-oil cosmetic of the present invention also may include a polyhydric alcohol-in-oil cosmetic that is substantially free of water. The form of the present cosmetic may be selected from various forms such as a liquid form, a milky lotion form, a cream form, a solid form, a paste form, a gel form, a powder form, a pressed form, a multi-layered form, a mousse form, a spray form, a stick form, and a pencil form.

The present cosmetic may contain various components used in conventional cosmetics as long as the effects of the present invention are not impaired. For example, it may include (1) an oil agent other than component (E), (2) powder other than component (C), (3) a film-forming agent, (4) a UV absorbing and scattering agent, and (5) other additives. These may be used alone or in an appropriate combination of two or more kinds.

### (1) Oil Agent

The oil agent may be solid, semi-solid, or liquid at room temperature (25° C.). Examples thereof include silicone oils, silicone waxes, natural animal and vegetable oils and fats, semi-synthetic oils and fats, hydrocarbon oils, higher alcohols, fatty acids, ester oils, fluorine-based oil agents, and UV absorbers. The amount of the oil agent in the cosmetic is not particularly limited, but is 1% to 95% by mass, preferably 5% to 65% by mass, or further preferably 5% to 30% by mass.

### Silicone Oils

Examples of the silicone oils include straight-chain or branched organopolysiloxanes having a viscosity ranging from low viscosity to high viscosity, such as dimethicone, alkyl-modified silicone, phenyl trimethicone, diphenyl dimethicone, diphenylsiloxy phenyl trimethicone, tetraphenyl dimethyl disiloxane, and methylhydrogen polysiloxane; amino-modified organopolysiloxanes such as amodimethicone and aminopropyl dimethicone; pyrrolidone-modified organopolysiloxanes such as PCA dimethicone; silicone rubbers such as PCA-modified organopolysiloxane, dimethylpolysiloxane gum having a high degree of polymerization, amino-modified organopolysiloxane gum, and dimethylsiloxane/methylphenylsiloxane copolymer gum; low-viscosity organopolysiloxane solutions of silicone gums and rubbers; amino-acid-modified silicones; fluorine-modified silicones; and silicone resins and their dissolved products.

Examples of commercially available silicone oils include KF-96A-6cs, KF-54, KF-54HV, and KF-56A, manufactured by Shin-Etsu Chemical Co., Ltd.

### Solid Oily Component

When solidification of the present cosmetic is desired, an oily component, which is solid at 25° C., is preferably blended into the cosmetic. The oily component, which is solid at 25° C., preferably has a melting point of 40° C. or more, or further preferably has a melting point of 60° C. to 110° C. Examples thereof include waxes, hydrocarbons, esters, higher alcohols, and higher fatty acids. It is not particularly limited and may be an ingredient that may be blended into conventional cosmetics. More specifically, examples thereof include vegetable waxes such as Copernicia cerifera (carnauba) wax, Saccharum officinarum (sugarcane) wax, Euphorbia cerifera (candelilla) wax, purified Euphorbia cerifera (candelilla) wax, rice bran wax, Japan wax, Simmondsia chinensis (jojoba) seed wax, kapok wax, Oryza sativa (rice) bran wax, Myrica cerifera (bayberry) fruit wax, Butyrospermum parkii (shea) butter, Theobroma cacao (cocoa) seed butter, Rhus succedanea fruit wax, montan wax, and hydrogenated castor oil isostearate; animal waxes such as beeswax, adeps bovis, beef bone tallow, adeps suillus, horse fat, mutton tallow, lanolin, Chinese wax, shellac wax, and spermaceti; semi-synthetic waxes such as lanolin esters, lanolin fatty acid esters, and beeswax acid esters; hydrogenated oils such as hydrogenated castor oil and hydrogenated coconut oil; hydrocarbon waxes such as solid paraffin, polyethylene, ceresin, ozokerite, and microcrystalline wax; wax esters such as synthetic beeswax; amino acid stearyl alcohols such as dioctyldodecyl lauroyl glutamate, dioctyldodecyl lauroyl glutamate and dioctyldodecyl lauroyl glutamate; fatty acids such as stearic acid and behenic acid; and silicone waxes such as silicone acrylate resins of silicone acrylate graft or block copolymers (silicone acrylate graft copolymers KP-561P, KP-562P, and the like, manufactured by Shin-Etsu Chemical Co., Ltd.); and derivatives thereof. One or more kinds selected from these are preferable.

### Natural Animal and Vegetable Oil Agents and Semi-Synthetic Oil Agents

Examples of the natural animal and vegetable oil agents and semi-synthetic oil agents include germ oils such as Persea gratissima (avocado) oil, Linum usitatissimum (linseed) seed oil, Prunus amygdalus dulcis (sweet almond) oil, Perilla frutescens seed oil, Olea europaea (olive) fruit oil, Torreya californica (California nutmeg) oil, Cymbopogon nardus (citronella) oil, Torreya nucifera seed oil, Prunus armeniaca (apricot) kernel oil, Triticum vulgare (wheat) germ oil, Sesamum indicum (sesame) seed oil, Triticum vulgare (wheat) germ oil, Oryza sativa (rice) germ oil, Oryza sativa (rice) bran oil, Camellia kissi seed oil, Carthamus tinctorius (safflower) seed oil, Glycine max (soybean) oil, Camellia sinensis seed oil, Camellia japonica seed oil, Oenothera biennis (evening primrose) oil, Brassica campestris (rapeseed) oil, Zea mays (corn) germ oil and Triticum vulgare (wheat) germ oil; natural vegetable oils such as Prunus armeniaca (apricot) kernel oil, Elaeis guineensis (palm) oil, Elaeis guineensis (palm) kernel oil, Ricinus communis (castor) seed oil, Helianthus annuus (sunflower) seed oil, Vitis vinifera (grape) seed oil, Simmondsia chinensis (jojoba) seed oil, Macadamia integrifolia seed oil, Limnanthes alba (meadowfoam) seed oil, Gossypium herbaceum (cotton) seed oil, Cocos nucifera (coconut) oil, and Arachis hypogaea (peanut) oil; natural animal oils such as shark liver oil, cod liver oil, fish liver oil, turtle oil, mink oil, and egg oil; and semi-synthetic oils and fats such as hydrogenated coconut oil and lanolin oil.

### Hydrocarbon Oils

Examples of the hydrocarbon oils include straight-chain or branched hydrocarbon oils. More specifically, examples thereof include alkanes such as hydrogenated polyisobutene, squalane, mineral oil, coconut alkanes, and C13-15 alkanes.

### Higher Alcohols

Examples of the higher alcohols include straight-chain saturated alcohols having 6 or more carbon atoms, such as lauryl alcohol, hexyldecanol, oleyl alcohol, isostearyl alcohol, octyldodecanol, decyltetradecanol, myristyl alcohol, cetyl alcohol, stearyl alcohol, and behenyl alcohol; and batyl alcohol. Other examples thereof include sterols such as cholesterol, beta-sitosterol, phytosterols, and lanosterol.

### Ester Oils

Examples of the ester oils include diisobutyl adipate; dihexyldecyl adipate; diheptylundecyl adipate; n-alkyl glycol monoisostearates such as isostearyl isostearate; isocetyl isostearate; trimethylolpropane triisostearate; glycol diethylhexanoate; cetyl ethylhexanoate; trimethylolpropane triethylhexanoate; pentaerythrityl tetraethylhexanoate; octyldodecyl esters such as octyldodecyl stearoyl stearate; oleyl oleate; octyldodecyl oleate; decyl oleate; neopentyl glycol diethylhexanoate; neopentyl glycol dicaprate; diisostearyl malate; triethyl citrate; diethylhexyl succinate; amyl acetate; ethyl acetate; butyl acetate; isocetyl stearate; butyl stearate; diisopropyl sebacate; diethylhexyl sebacate; cetyl lactate; myristyl lactate; isononyl isononanoate; isotridecyl isononanoate; palmitates such as isopropyl palmitate, ethylhexyl palmitate, and hexyldecyl palmitate; myristates such as cholesteryl, isopropyl myristate, octyldodecyl myristate, and myristyl myristate; ethylhexyl laurate; hexyl laurate; dioctyldodecyl lauroyl glutamate; isoproyl lauroyl sarcosinate; and coco-caprylate/caprate.

Among the ester oils, examples of glyceride oils include triethylhexanoin, caprylic/capric triglyceride, cocoglycerides, caprylic/capric/succinic triglyceride, and caprylic/capric glycerides.

### Fluorine-Based Oil Agents

Examples of the fluorine-based oil agents include perfluorodecalin, perfluorononyl dimethicone, and perfluoromethylcyclopentane.

### UV Absorbers

Examples of the UV absorbers include benzophenone-1, benzophenone-2, benzophenone-3, benzophenone-4, benzophenone-5, benzophenone-6, benzophenone-9, homosalate, octocrylene, butyl methoxydibenzoylmethane, ethylhexyl salicylate, diethylamino hydroxybenzoyl hexyl benzoate, polysilicone-15, ethylhexyl dimethoxybenzylidene dioxoimidazolidine propionate, terephthalylidene dicamphor sulfonic acid, ethylhexyl triazone, isopentyl trimethoxycinnamate trisiloxane, drometrizole trisiloxane, ethylhexyl dimethyl PABA, isopropyl methoxycinnamate, ethylhexyl methoxycinnamate, bis-ethylhexyloxyphenol methoxyphenyl triazine, phenylbenzimidazole sulfonic acid, methylene bis-benzotriazolyl tetramethylbutylphenol, glyceryl ethylhexanoate dimethoxycinnamate, glyceryl PABA, diisopropyl methyl cinnamate, cinoxate, and ethylhexyl dimethoxybenzylidene dioxoimidazolidine propionate. It is also possible to use a UVA absorber (examples thereof include diethylamino hydroxybenzoyl hexyl benzoate) in combination with a UVB absorber (examples thereof include ethylhexyl methoxycinnamate), and each of them may be optionally combined.

(2) Powder Other than Component (C)

The powder other than component (C), that is, powder whose amount of absorption of the hydrocarbon oil and the ester oil is less than 30 parts by mass, relative to 100 parts by mass, may be blended into the cosmetic. More specifically, powders having low amounts of oil absorption, such as the color pigments, inorganic powders, metal powders, organic powders, and inorganic-organic composite powders exemplified in (C), are exemplified.

Specific examples of (2) include, as commercially available products, the KTP-09 series (KTP-09W, KTP-09R, KTP-09Y, and KTP-09B), KMP-590, and KMP-591, manufactured by Shin-Etsu Chemical Co., Ltd. They are color pigments and silicone resin particles (polyorganosilsesquioxane resin particles with a three-dimensional network structure) subjected to a surface treatment.

### (3) Film-Forming Agent

The film-forming agent is blended into the cosmetic mainly for the purpose of further maintaining durability in terms of cosmetic effects. The film-forming agent is not particularly limited. The film-forming agent is preferably a silicone-based composition in view of the impartation of water repellency. More specifically, trimethylsiloxysilicate, silicone acrylate coating agents, silicone-modified norbornene, silicone-modified pullulan, silicone-modified polyvinyl alcohols, and the like may be used.

Examples of a silicone-based composition as a film-forming agent include trimethylsiloxysilicate, acrylates/dimethicone copolymer, norbornene/tris(trimethylsiloxy) silylnorbornene copolymer, and trimethylsiloxysilylcarbamoyl pullulan.

The film-forming agent may be dissolved in an oil agent which is liquid at room temperature in advance and then blended into the cosmetic. The liquid oil agent may be component (F) or a liquid silicone oil, hydrocarbon oil, ester oil, natural animal or vegetable oil, semi-synthetic oil, fluorine-based oil, or the like in (1) the oil agent of the optional components.

Specific examples of commercially available silicone film-forming agents include KF-7312J, KP-545, KP-549, KP-543, NBN-30-ID, TSPL-30-ID, and TSPL-30-D5, manufactured by Shin-Etsu Chemical Co., Ltd.

### (4) UV Absorbing and Scattering Agent

Examples of the UV absorbing and scattering agent include particles that absorb and scatter UV rays, such as titanium oxide fine particles, iron-containing titanium oxide fine particles, zinc oxide fine particles, cerium oxide fine particles, and composites thereof. A dispersion in which these particles that absorb and scatter UV rays are dispersed in an oil agent in advance may also be used. Specific examples of the dispersion in which particles that absorb and scatter UV rays are dispersed in an oil agent in advance include the SPD series (product name), particularly, SPD-T5, SPD-T5L, SPD-Z5, SPD-Z5L, SPD-T6, SPD-Z6, SPD-T7, and SPD-Z7L, manufactured by Shin-Etsu Chemical Co., Ltd.

### (5) Other Additives

Examples of the other additives include oil-soluble gelling agents, preservatives/antiseptics, antiperspirants, perfumes, salts, antioxidants, pH adjusters, chelating agents, refreshing agents, anti-inflammatory agents, skin-beautifying components (skin-lightening agents, cell activators, rough-skin-improving agents, blood circulation promoters, skin astringents, anti-seborrheics, and the like), vitamins, amino acids, nucleic acids, hormones, and inclusion compounds.

### Oil-Soluble Gelling Agents

Examples of the oil-soluble gelling agents include lime soaps such as aluminum stearate, magnesium stearate, and zinc myristate; amino acid derivatives such as lauroyl glutamic acid and α, γ-di-n-butylamine; dextrin fatty acid esters such as dextrin palmitate, dextrin isostearate, dextrin myristate, stearoyl inulin, and dextrin palmitate/ethylhexanoate; sucrose fatty acid esters such as sucrose palmitate and sucrose stearate; fructooligosaccharide fatty acid esters such as fructooligosaccharide stearate and fructooligosaccharide 2-ethylhexanoate; benzylidene derivatives of sorbitol such as monobenzylidene sorbitol and dibenzylidene sorbitol; organically modified clay minerals such as disteardimonium hectorite, stearalkonium hectorite, and hectorite; and stearalkonium bentonite.

### Preservatives/Antiseptics

Examples of the preservatives/antiseptics include p-hydroxybenzoate alkyl ester, benzoic acid, sodium benzoate, sorbic acid, potassium sorbate, phenoxyethanol, imidazolidinyl urea, salicylic acid, o-cymen-5-ol, phenol, p-chloro-m-cresol, hexachlorophene, benzalkonium chloride, chlorhexidine chloride, trichlorocarbanilide, iodopropynyl butylcarbamate, polylysine, photosensitizers, silver, and plant extracts.

### Antiperspirants

Examples of the antiperspirants include aluminum hydroxy-halides such as aluminum chlorohydrate; aluminum halides such as aluminum chloride; aluminum salts of allantoin; tannic acid; persimmon tannin; potassium alum; zinc oxide; zinc p-phenolsulfonate; burnt alum; aluminum zirconium tetrachlorohydrate; and aluminum zirconium trichlorohydrex GLY. In particular, as components that demonstrate strong effects, aluminum hydroxy-halides, aluminum halides, their complexes or mixtures with zirconyl oxy-halides and zirconyl hydroxy-halides (examples thereof include aluminum zirconium tetrachlorohydrate and aluminum zirconium trichlorohydrex GLY), and the like are preferable.

### Perfumes

Examples of the perfumes include natural perfumes and synthetic perfumes. Examples of the natural perfumes include plant-based perfumes isolated from flowers, leaves, wood, peels, and the like; and animal-based perfumes such as musk and civet. Examples of the synthetic perfumes include hydrocarbons such as monoterpenes; alcohols such as aliphatic alcohols and aromatic alcohols; aldehydes such as terpene aldehydes and aromatic aldehydes; ketones such as alicyclic ketones; esters such as terpene-based esters; lactones; phenols; oxides; nitrogen-containing compounds; and acetals.

### Salts

Examples of the salts include inorganic salts, organic acid salts, amine salts, and amino acid salts. Examples of inorganic salts include sodium salts, potassium salts, magnesium salts, calcium salts, aluminum salts, zirconium salts, zinc salts, and the like of inorganic acids such as hydrochloric acid, sulfuric acid, carbonic acid, and nitric acid. Examples of organic acid salts include salts of organic acids such as acetic acid, dehydroacetic acid, citric acid, malic acid, succinic acid, ascorbic acid, and stearic acid. Examples of amine salts and amino acid salts include salts of amines such as triethanolamine; and salts of amino acids such as glutamic acid. In addition, other examples of salts that may be used include salts of hyaluronic acid, chondroitin sulfate, and the like; aluminum zirconium glycine complexes; and neutral salts of acids and alkalis used in cosmetic formulations.

### Antioxidants

The antioxidants are not particularly limited. Examples thereof include carotenoids, ascorbic acid and its salts, ascorbyl stearate, tocophenols, tocopheryl acetate, tocopherols, 4-tert-butylphenol, BHA, BHT, phytic acid, ferulic acid, thiotaurine, aminoethanesulfinic acid, sulfite salts, erythorbic acid and its salts, chlorogenic acids, epicatechin, epigallocatechin, epigallocatechin gallate, apigenin, kaempferol, myricetin, and quercetin. The antioxidants may be used alone or in combination of two or more kinds.

### pH Adjusters

Examples of the pH adjusters include lactic acid, citric acid, glycolic acid, succinic acid, tartaric acid, DL-malic acid, potassium carbonate, sodium bicarbonate, and ammonium bicarbonate.

### Chelating Agents

Examples of the chelating agents include alanine, sodium salts of EDTA acid, sodium polyphosphate, sodium metaphosphate, and phosphoric acid.

### Refreshing Agents

Examples of the refreshing agents include L-menthol, camphor, and menthyl lactate.

### Anti-Inflammatory Agents

Examples of the anti-inflammatory agents include allantoin, glycyrrhizic acid and its salts, glycyrrhetinic acid, stearyl glycyrrhetinate, tranexamic acid, and azulene.

### Skin-Beautifying Components

Examples of the skin-beautifying components include skin-lightening agents such as placental extracts, arbutin, glutathione, and Saxifraga sarmentosa extracts; cell activators such as royal jelly, photosensitizers, cholesterol derivatives, and calf blood extracts; rough-skin-improving agents; blood circulation promoters such as 4-hydroxy-3-methoxybenzyl nonylic acid amide, benzyl nicotinate, β-butoxyethyl nicotinate, capsaicin, zingerone, cantharides tincture, ichthammol, caffeine, tannic acid, α-borneol, tocopheryl nicotinate, inositol hexaniacinate, cyclandelate, cinnarizine, tolazoline, acetylcholine, verapamil, cepharanthine, and γ-oryzanol; skin astringents such as zinc oxide and tannic acid; and anti-seborrheics such as sulfur and thianthol.

### Vitamins

Examples of the vitamins include vitamin A, such as vitamin A oils, retinol, retinyl acetate, and retinyl palmitate; vitamin B2, such as riboflavin, riboflavin butyrate, and flavin adenine dinucleotide; vitamin B6, such as pyridoxine HCl, pyridoxine dicaprylate, and pyridoxine tripalmitate; vitamin B, such as vitamin B12, vitamin B15, and their derivatives; vitamin C, such as L-ascorbic acid, L-ascorbyl dipalmitate, disodium L-ascorbyl sulfate, and dipotassium L-ascorbyl phosphate; vitamin D, such as ergocalciferol and cholecalciferol; vitamin E, such as α-tocopherol, β-tocopherol, γ-tocopherol, DL-α-tocopheryl acetate, DL-α-tocopheryl nicotinate, and DL-α-tocopheryl succinate; niacin, such as nicotinic acid, benzyl nicotinate, and niacinamide; vitamin H; vitamin P; pantothenic acids such as calcium pantothenate, D-panthenol, panthenyl ethyl ether, and panthenyl ethyl ether acetate; and biotin.

### Amino Acids

Examples of the amino acids include glycine, valine, leucine, isoleucine, serine, threonine, phenylalanine, arginine, lysine, aspartic acid, glutamic acid, cystine, cysteine, methionine, and tryptophan.

### Nucleic Acids

Examples of the nucleic acids include deoxyribonucleic acid.

### Hormones

Examples of the hormones include estradiol and ethinylestradiol.

### Inclusion Compounds

Examples of the inclusion compounds include cyclodextrin.

### EXAMPLES

The present invention will be explained below in further detail with reference to a series of the Synthesis examples, the Examples and the Comparative Examples, though the present invention is in no way limited by these Examples.

In the following examples, kinematic viscosity is the value at 25° C. determined with a Cannon-Fenske viscometer according to JIS Z8803:2011. The unit “%” for concentration and content refers to “mass %”.

The long-chain alkyl group amount % means the ratio of the moles of long-chain alkyl group amount bonded to the silicon atom to the total moles of all substituent groups bonded to the silicon atom, excluding hydrogen atoms bonded to the silicon atom.

### Synthesis Example 1

In a glass beaker having a volume of 1 L, were placed 400 g of organohydrogen polysiloxane having 22.2 mol % of long-chain alkyls and a kinematic viscosity of 110 mm²/s, represented by the following formula (1),

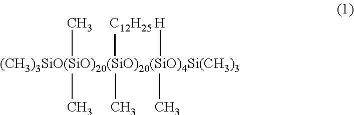

114 g of methyl vinyl polysiloxane having no long-chain alkyl groups, represented by the following formula (2), and having a kinematic viscosity of 10 mm²/s (such amount that the ratio of the number of vinyl groups in the methyl vinyl polysiloxane relative to one hydrosilyl group in the organohydrogen polysiloxane is 0.96),

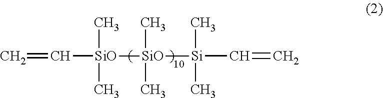

and 0.1 g of DL-α-tocopherol and were mixed and stirred with a homomixer at 2,000 rpm. Into the resulting mixed solution, were added 1.0 g of polyoxyethylene lauryl ether (Ethylene oxide addition mole number is 9 mols) and 190 g of water and were stirred with a homomixer at 6,000 rpm to obtain an O/W emulsion, which was found to have increased viscosity. Stirring was further continued for 15 minutes. Then, 274 g of water was added thereto while stirring at 2,000 rpm to obtain a homogeneous white emulsion.

This emulsion was transferred to a glass flask having a volume of 1 L, which was equipped with a stirrer having an anchor stirring blade, and the temperature was adjusted to 15° C. to 20° C. Then, 18 g of polyoxyethylene lauryl ether (Ethylene oxide addition mole number is 9 mols) was added thereto and dissolved in the emulsion by stirring for 30 minutes. Then, a mixed dissolved product of 2.4 g of an isododecane solution of a platinum- and vinyl-group-containing disiloxane complex (platinum content is 0.5%) and 1.0 g of polyoxyethylene lauryl ether (Ethylene oxide addition mole number is 9 mols) was added thereto under stirring, and the resultant was stirred at the same temperature for 12 hours to obtain a water dispersion of silicone rubber particles.

The shape of the silicone rubber particles was observed by an optical microscope to be confirmed spherical. The volume average particle size thereof determined with a particle size distribution measuring device “Multisizer 3” (product name; manufactured by Beckman Coulter, Inc.) was 12 μm.

### Synthesis Example 2

In a glass beaker having a volume of 1 L, were placed 400 g of organohydrogen polysiloxane having 10.5 mol % of long-chain alkyls, represented by the following formula (3), and having a kinematic viscosity of 130 mm²/s,

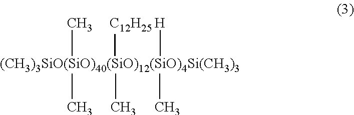

113 g of methyl vinyl polysiloxane having no long-chain alkyl groups, represented by the aforesaid formula (2), and having a kinematic viscosity of 10 mm²/s (such amount that the ratio of the number of vinyl groups in the methyl vinyl polysiloxane relative to one hydrosilyl group in the organohydrogen polysiloxane is 0.91), and 0.1 g of DL-α-tocopherol and were mixed and stirred with a homomixer at 2,000 rpm. Into the resulting mixed solution, were added 1.0 g of polyoxyethylene lauryl ether (Ethylene oxide addition mole number is 9 mols) and 190 g of water and were stirred with a homomixer at 6,000 rpm to obtain an O/W emulsion, which was found to have increased viscosity. Stirring was further continued for 15 minutes. Then, 275 g of water was added thereto while stirring at 2,000 rpm to obtain a homogeneous white emulsion.

This emulsion was transferred to a glass flask having a volume of 1 L, which was equipped with a stirrer having an anchor stirring blade, and the temperature was adjusted to 15° C. to 20° C. Then, 18 g of polyoxyethylene lauryl ether (Ethylene oxide addition mole number is 9 mols) was added thereto and dissolved in the emulsion by stirring for 30 minutes. Then, a mixed dissolved product of 2.4 g of an isododecane solution of a platinum- and vinyl-group-containing disiloxane complex (platinum content is 0.5%) and 1.0 g of polyoxyethylene lauryl ether (Ethylene oxide addition mole number is 9 mols) was added thereto under stirring, and the resultant was stirred at the same temperature for 12 hours to obtain a water dispersion of silicone rubber particles.

The shape of the silicone rubber particles was observed by an optical microscope to be confirmed spherical. The volume average particle size thereof determined with a particle size distribution measuring device “Multisizer 3” (product name; manufactured by Beckman Coulter, Inc.) was 12 μm.

### Synthesis Example 3

In a glass beaker having a volume of 1 L, were placed 467 g of organohydrogen polysiloxane having 22.2 mol % of long-chain alkyls, represented by the aforesaid formula (1), and having a kinematic viscosity of 110 mm²/s, 133 g of methyl vinyl polysiloxane having no long-chain alkyl groups, represented by the aforesaid formula (2), and having a kinematic viscosity of 10 mm²/s (such amount that the ratio of the number of vinyl groups in the methyl vinyl polysiloxane relative to one hydrosilyl group in the organohydrogen polysiloxane is 0.96), and 0.1 g of DL-α-tocopherol and were mixed and stirred with a homomixer at 2,000 rpm. Into the resulting mixed solution, were added 0.5 g of polyoxyethylene stearyl ether (Ethylene oxide addition mole number is 6 mols), 1.6 g of polyoxyethylene stearyl ether (Ethylene oxide addition mole number is 20 mols), 10 g of phenoxyethanol and 200 g of water and were stirred with a homomixer at 6,000 rpm to obtain an O/W emulsion, which was found to have increased viscosity. Stirring was further continued for 15 minutes. Then, 164 g of water was added thereto while stirring at 2,000 rpm to obtain a homogeneous white emulsion.

This emulsion was transferred to a glass flask having a volume of 1 L, which was equipped with a stirrer having an anchor stirring blade, and the temperature was adjusted to 15° C. to 20° C. Then, 5 g of polyoxyethylene stearyl ether (Ethylene oxide addition mole number is 6 mols) and 14 g of polyoxyethylene stearyl ether (Ethylene oxide addition mole number is 20 mols) were added thereto and dissolved in the emulsion by stirring for 30 minutes. Then, a mixed dissolved product of 2.5 g of an isododecane solution of a platinum- and vinyl-group-containing disiloxane complex (platinum content is 0.5%), 1.1 g of polyoxyethylene sorbitan tristearate (Ethylene oxide addition mole number is 20 mols) and 1.1 g of polyoxyethylene sorbitan monostearate (Ethylene oxide addition mole number is 20 mols) was added thereto under stirring, and the resultant was stirred at the same temperature for 12 hours to obtain a water dispersion of silicone rubber particles.

The shape of the silicone rubber particles was observed by an optical microscope to be confirmed spherical. The volume average particle size thereof determined with a particle size distribution measuring device “Multisizer 3” (product name; manufactured by Beckman Coulter, Inc.) was 5 μm.

### Synthesis Example 4

In a glass beaker having a volume of 1 L, were placed 400 g of organohydrogen polysiloxane having 22.2 mol % of long-chain alkyls, represented by the aforesaid formula (1), and having a kinematic viscosity of 110 mm²/s, 114 g of methyl vinyl polysiloxane having no long-chain alkyl groups, represented by the aforesaid formula (2), and having a kinematic viscosity of 10 mm²/s (such amount that the ratio of the number of vinyl groups in the methyl vinyl polysiloxane relative to one hydrosilyl group in the organohydrogen polysiloxane is 0.96), and 0.1 g of DL-α-tocopherol and were mixed and stirred with a homomixer at 2,000 rpm. Into the resulting mixed solution, were added 1.0 g of polyoxyethylene lauryl ether (Ethylene oxide addition mole number is 9 mols) and 100 g of water and were stirred with a homomixer at 6,000 rpm to obtain an O/W emulsion, which was found to have increased viscosity. Stirring was further continued for 15 minutes. Then, 382 g of water was added thereto while stirring at 2,000 rpm to obtain a homogeneous white emulsion.

This emulsion was transferred to a glass flask having a volume of 1 L, which was equipped with a stirrer having an anchor stirring blade, and the temperature was adjusted to 15° C. to 20° C. Then, a mixed dissolved product of 1.6 g of an isododecane solution of a platinum- and vinyl-group-containing disiloxane complex (platinum content is 0.5%), 0.6 g of polyoxyethylene lauryl ether (Ethylene oxide addition mole number is 9 mols) and 0.4 g of polyoxyethylene lauryl ether (Ethylene oxide addition mole number is 4 mols) was added thereto under stirring, and the resultant was stirred at the same temperature for 12 hours to obtain a water dispersion of silicone rubber particles.

The shape of the silicone rubber particles was observed by an optical microscope to be confirmed spherical. The volume average particle size thereof determined with a particle size distribution measuring device “Multisizer 3” (product name; manufactured by Beckman Coulter, Inc.) was 12 μm.

Then, 876 g of the resulting water dispersion of the silicone rubber particles was transferred to a glass flask having a volume of 3 L, which was equipped with a stirrer having an anchor stirring blade, and 1, 988 g of water and 57 g of 28% ammonia water were added thereto. The pH of the liquid at this time was 11.2. After the temperature was adjusted to 5° C. to 10° C., a mixed dissolved product of 76.8 g of methyltrimethoxysilane and 2.1 g of γ-aminopropyltrimethoxysilane (such amount that the amount of polyorganosilsesquioxane after hydrolysis and condensation reactions would be 8.7 parts by mass, relative to 100 parts by mass of the silicone rubber particles) was added dropwise over 30 minutes, and the resultant was further stirred for 1 hour. During this time, the temperature of the liquid was maintained at 5° C. to 10° C. Then, the resultant was heated to 55° C. to 60° C. and stirred for 1 hour with its temperature maintained to complete the hydrolysis and condensation reactions of the methoxysilanes.

The liquid obtained by subjecting the methoxysilanes to the hydrolysis and condensation reactions was filtered and dehydrated with a pressure filter. The dehydrated product was transferred to a stainless steel tray and dried in a hot air circulation dryer at a temperature of 105° C., and the dried product was crushed with a jet mill to obtain fine particles with fluidity. The fine particles were observed with an electron microscope, and it was confirmed that the fine particles were spherical particles whose surface was covered with granular products having a particle size of about 100 nm and that the fine particles were silicone fine particles obtained by coating silicone rubber particles with polyorganosilsesquioxane. The resulting silicone rubber powder coated with silicone resin was dispersed in water with a surfactant and the volume average particle size was determined with a “Multisizer 3” to be confirmed that the volume average particle size was 13 μm.

### Comparative Synthesis Example 1

In a glass beaker having a volume of 1 L, were placed 19 g of methylhydrogenpolysiloxane represented by the following formula (4) and having a kinematic viscosity of 30 mm²/s,

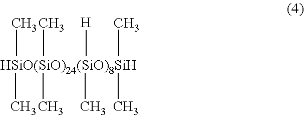

500 g of methyl vinyl polysiloxane having no long-chain alkyl groups, represented by the following formula (5), and having a kinematic viscosity of 580 mm²/s (such amount that the ratio of the number of vinyl groups in the methyl vinyl polysiloxane relative to one hydrosilyl group in the organohydrogen polysiloxane is 0.95),

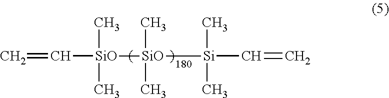

and 0.1 g of DL-α-tocopherol and were mixed and stirred with a homomixer at 2,000 rpm. Into the resulting mixed solution, were added 1.2 g of polyoxyethylene lauryl ether (Ethylene oxide addition mole number is 9 mols) and 100 g of water and were stirred with a homomixer at 6,000 rpm to obtain an O/W emulsion, which was found to have increased viscosity. Stirring was further continued for 15 minutes. Then, 377 g of water was added thereto while stirring at 2,000 rpm to obtain a homogeneous white emulsion.

This emulsion was transferred to a glass flask having a volume of 1 L, which was equipped with a stirrer having an anchor stirring blade, and the temperature was adjusted to 15° C. to 20° C. Then, a mixed dissolved product of 0.8 g of an isododecane solution of a platinum- and vinyl-group-containing disiloxane complex (platinum content is 0.5%) and 1.8 g of polyoxyethylene lauryl ether (Ethylene oxide addition mole number is 9 mols) was added thereto under stirring, and the resultant was stirred at the same temperature for 12 hours to obtain a water dispersion of silicone rubber particles.

The shape of the silicone rubber particles was observed by an optical microscope to be confirmed spherical. The volume average particle size thereof determined with a “Multisizer 3” (product name; manufactured by Beckman Coulter, Inc.) particle size distribution measuring device was 12 μm.

### [Measurement of the Hardness of the Silicone Rubber Particles]

Each of the isododecane solutions above (platinum content=0.5%) of organohydrogen polysiloxane, methyl vinyl polysiloxane, and the platinum- and vinyl-group-containing disiloxane complex used in Synthesis Examples 1 to 4 and the Comparative Synthesis Examples was mixed with the blending ratio described respectively in Synthesis Examples 1 to 4 and the Comparative Synthesis Examples and poured into an aluminum Petri dish so as to have a thickness of 10 mm. After the resultant was left to stand at 25° C. for 24 hours, it was heated in a thermostatic chamber at 50° C. for 1 hour to obtain a silicone rubber that does not exhibit any stickiness. The hardness of the silicone rubber was determined with an ASKER Durometer Type C specified in SRIS 0101. The results are shown in Table 1.

### [Measurement of the Amount of Oil Absorption of the Silicone Rubber Particles]

Each of the isododecane solutions above (platinum content=0.5%) of organohydrogen polysiloxane, methyl vinyl polysiloxane, and the platinum- and vinyl-group-containing disiloxane complex used in Synthesis Examples 1 to 4 and the Comparative Synthesis Examples above was mixed with the blending ratio described respectively in Synthesis Examples 1 to 4 and the Comparative Synthesis Examples above and poured into a PTFE resin tray (80 mm×130 mm) so as to have a thickness of about 1 mm. After the resultant was left to stand at 25° C. for 24 hours, it was heated in a thermostatic chamber at 50° C. for 1 hour to obtain a silicone rubber sheet. The resulting sheet was cut to a size of an approximately 30 mm×30 mm square to obtain a test piece, the mass of the test piece was then measured, and the test piece was then immersed in the oily substance shown in Table 1 at 25° C. for 24 hours. The test piece, which absorbed the oily substance and swelled, was removed, and the oily substance on the surface was wiped off with tissue paper. Then, the mass of the test piece was measured. Table 1 shows the amount of the oily substance absorbed by the silicone rubber sheet (amount of oil absorption), which is shown as a value relative to 100 g of the silicone rubber.

TABLE 1

| | | | | | | Comparative |
|---|---|---|---|---|---|---|
| | | Synthesis | Synthesis | Synthesis | Synthesis | Synthesis |
| | | Example1 | Example2 | Example3 | Example4 | Example1 |
| Oil | Squalane | 160 | 70 | 160 | 160 | 10 or less |
| absorption | Ethylhexyl | 150 | 70 | 150 | 150 | 20 |
| per 100 g | palmitate | | | | | |
| of silicone | Liquid | 70 | 40 | 70 | 70 | 10 or less |
| rubber (g) | paraffin | | | | | |
| | Isocetyl | 110 | 60 | 110 | 110 | 10 or less |
| | myristate | | | | | |
| | Isocetyl | 100 | 50 | 100 | 100 | 10 or less |
| | isostearate | | | | | |

| | | | | | |
|---|---|---|---|---|---|
| Silicone rubber | 44 | 53 | 44 | 44 | 55 |
| hardness, Asker C | | | | | |

### Examples 1 to 13, Comparative Examples 1 to 6

Water-in-oil water-break cosmetics were prepared by mixing the ingredients according to the compositions described in the following Tables 2, 3 and 4.

TABLE 2

| Composition, | | Example | | | | | | |
|---|---|---|---|---|---|---|---|---|
| part by mass | | 1 | 2 | 3 | 4 | 5 | 6 | 7 |
| Oil | (A) KSG-240 ^{(note 1)} | 3 | 3 | 2 | 3 | 4 | 3 | 3 |
| phase | (A) KSG-210 ^{(note 2)} | | | | | | | |
| | (F) KSG-15 ^{(note 3)} | 1 | 1 | 1 | 1 | 1 | 1 | 1 |
| | (F) KSG-19 ^{(note 4)} | | | | | | | |
| | (B) KF-6017 ^{(note 5)} | 0.2 | 0.2 | 0.2 | 0.2 | 0.2 | 0.1 | 0.5 |
| | Dimethicone, 6cs | | | | | | | |
| | (E) Cyclopentasiloxane | 8.8 | 8.8 | 9.8 | 8.8 | 7.8 | 8.9 | 8.5 |
| Aqueous | Glycerin | 8 | 8 | 8 | 8 | 8 | 8 | 8 |
| phase | BG, Butylene glycol | 4 | 4 | 4 | 4 | 4 | 4 | 4 |
| | Sodium citrate | 0.2 | 0.2 | 0.2 | 0.2 | 0.2 | 0.2 | 0.2 |
| | Sodium chloride | 1 | 1 | 1 | 1 | 1 | 1 | 1 |
| | (C) Water dispersion of | 5.8 | | | | | | |
| | synthesis example 1, | | | | | | | |
| | 51.4% ^{(note 6)} | | | | | | | |
| | (C) Water dispersion of | | 5.8 | | | | | |
| | synthesis example 2, | | | | | | | |
| | 51.3% ^{(note 6)} | | | | | | | |
| | (C) Water dispersion of | | | 5 | 5 | 5 | 5 | 5 |
| | synthesis example 3, 60% | | | | | | | |
| | ^{(note 6)} | | | | | | | |
| | (A) KF-6011 ^{(note 8)} | | | | | | | |
| | Purified water | balance | balance | balance | balance | balance | balance | balance |
| | Total, parts by mass | 100 | 100 | 100 | 100 | 100 | 100 | 100 |
| | Amount of silicone | 3 | 3 | 3 | 3 | 3 | 3 | 3 |
| | particles | | | | | | | |
| | Amount of aqueous | 84 | 84 | 84 | 84 | 84 | 84 | 84 |
| | components ^{(note 9)} | | | | | | | |
| Evaluation | Availability to create | B | B | B | B | B | B | B |
| | make-up | | | | | | | |
| | Viscosity stability | A | A | B | A | A | B | A |
| | Aesthetics | A | A | A | A | A | A | A |
| | Spreadability at the | A | B | B | A | B | B | B |
| | time of application | | | | | | | |
| | Appearance at the finish | A | B | A | A | B | A | A |
| | Stickiness at the finish | C | C | B | B | B | A | C |
| | Durability | B | C | B | B | B | B | B |

TABLE 3

| Composition, | | Example | | | | | |
|---|---|---|---|---|---|---|---|
| part by mass | | 8 | 9 | 10 | 11 | 12 | 13 |
| Oil | (A) KSG-240 ^{(note 1)} | 3 | 3 | 3 | 3 | | 3 |
| phase | (A) KSG-210 ^{(note 2)} | | | | | 3 | |
| | (F) KSG-15 ^{(note 3)} | 1 | 1 | 1 | 1 | | |
| | (F) KSG-19 ^{(note 4)} | | | | | 1 | |
| | (B) KF-6017 ^{(note 5)} | 0.2 | 0.2 | 0.2 | 0.2 | 0.2 | 0.2 |
| | Dimethicone, 6cs | | | | | 8.8 | |
| | (E) Cyclopentasiloxane | 8.8 | 8.8 | 8.8 | 5.8 | | 9.8 |
| Aqueous | Glycerin | 8 | 8 | 8 | 8 | 8 | 8 |
| phase | BG, Butylene glycol | 4 | 4 | 4 | 4 | 4 | 4 |
| | Sodium citrate | 0.2 | 0.2 | 0.2 | 0.2 | 0.2 | 0.2 |
| | Sodium chloride | 1 | 1 | 1 | 1 | 1 | 1 |
| | (C) Water dispersion of | | | | | | |
| | synthesis example 1, | | | | | | |
| | 51.4% ^{(note 6)} | | | | | | |
| | (C) Water dispersion of | | | | | | |
| | synthesis example 2, | | | | | | |
| | 51.3% ^{(note 6)} | | | | | | |
| | (C) Water dispersion of | 3 | 10 | | 5 | 5 | 5 |
| | synthesis example 3, 60% | | | | | | |
| | ^{(note 6)} | | | | | | |
| | (A) Silicone rubber powder | | | 3 | | | |
| | coated with silicone resin. | | | | | | |
| | of synthesis example 4 | | | | | | |
| | (B) KF-6011 ^{(note 8)} | | | 0.5 | | | |
| | Purified water | balance | balance | balance | balance | balance | balance |
| | Total, parts by mass | 100 | 100 | 100 | 100 | 100 | 100 |
| | Amount of silicone | 1.8 | 6 | 3 | 3 | 3 | 3 |
| | particles | | | | | | |
| | Amount of aqueous | 85.2 | 81 | 84 | 87 | 84 | 84 |
| | components ^{(note 9)} | | | | | | |
| Evaluation | Availability to create | B | B | B | B | B | B |
| | make-up | | | | | | |
| | Viscosity stability | A | C | C | C | C | C |
| | Aesthetics | A | A | A | A | B | A |
| | Spreadability at the | B | B | B | C | A | A |
| | time of application | | | | | | |
| | Appearance at the finish | B | B | A | A | B | A |
| | Stickiness at the finish | B | C | B | B | B | B |
| | Durability | C | A | B | B | C | B |

TABLE 4

| Composition, | | Comparative Example | | | | | |
|---|---|---|---|---|---|---|---|
| part by mass | | 1 | 2 | 3 | 4 | 5 | 6 |
| Oil | (A) KSG-240 ^{(note 1)} | | 3 | 3 | 3 | 3 | 3 |
| phase | (F) KSG-15 ^{(note 3)} | 1 | 1 | 1 | 1 | 1 | 1 |
| | (B) KF-6017 ^{(note 5)} | 0.2 | | 0.2 | 0.2 | 0.2 | 0.2 |
| | (E) Cyclopentasiloxane | 11.8 | 9 | 8.8 | 8.8 | 8.8 | 8.8 |
| | (C) Silicone rubber powder | | | | | | 3 |
| | coated with silicone resin. | | | | | | |
| | of synthesis example 4 | | | | | | |
| Aqueous | Glycerin | 8 | 8 | 8 | 8 | 8 | 8 |
| phase | BG, Butylene glycol | 4 | 4 | 4 | 4 | 4 | 4 |
| | Sodium citrate | 0.2 | 0.2 | 0.2 | 0.2 | 0.2 | 0.2 |
| | Sodium chloride | 1 | 1 | 1 | 1 | 1 | 1 |
| | (C) Water dispersion of | 5 | 5 | | | | |
| | synthesis example 3, 60% | | | | | | |
| | ^{(note 6)} | | | | | | |
| | Water dispersion of | | | | 5.8 | | |
| | comparative synthesis | | | | | | |
| | example 1, 51.9% ^{(note 6)} | | | | | | |
| | KMP-591 ^{(note 7)} | | | | | 3 | |
| | (B) KF-6011 ^{(note 8)} | | | | | 0.5 | |
| | Purified water | balance | balance | balance | balance | balance | balance |
| | Total, parts by mass | 100 | 100 | 100 | 100 | 100 | 100 |
| | Amount of silicone | 3 | 3 | 0 | 3 | 3 | 3 |
| | particles | | | | | | |
| | Amount of aqueous | 84 | 84 | 87 | 84 | 84 | 84 |
| | components (note 9) | | | | | | |
| Evaluation | Availability to create | D | B | B | B | B | B |
| | make-up | | | | | | |
| | Viscosity stability | | D | A | D | D | B |
| | Aesthetics | | A | B | B | B | E |
| | Spreadability at the | | D | C | B | B | E |
| | time of application | | | | | | |
| | Appearance at the finish | | A | C | C | C | C |
| | Stickiness at the finish | | B | B | B | B | C |
| | Durability | | B | D | D | D | B |

(Note 1) Mixture of 75% to 85% cyclopentasiloxane+15% to 25% dimethicone/PEG-10/15 crosspolymer (manufactured by Shin-Etsu Chemical Co., Ltd.)
(Note 2) Mixture of 70% to 80% dimethicone (6cs)+20% to 30% dimethicone/PEG-10/15 crosspolymer (manufactured by Shin-Etsu Chemical Co., Ltd.)
(Note 3) Mixture of 90% to 96% cyclopentasiloxane+4% to 10% dimethicone/vinyl dimethicone crosspolymer (manufactured by Shin-Etsu Chemical Co., Ltd.)
(Note 4) Mixture of 80% to 90% dimethicone (6cs)+10% to 20% dimethicone/vinyl dimethicone crosspolymer (manufactured by Shin-Etsu Chemical Co., Ltd.) (Note 5) PEG-10 dimethicone (manufactured by Shin-Etsu Chemical Co., Ltd.)
(Note 6) The percentage in parentheses is the concentration of silicone rubber particles in the water dispersion.
(Note 7) Polymethylsilsesquioxane (manufactured by Shin-Etsu Chemical Co., Ltd.)
(Note 8) PEG-11 methyl ether dimethicone (manufactured by Shin-Etsu Chemical Co., Ltd.)
(Note 9) The amount of the aqueous component is the amount obtained by subtracting the amount of silicone particles from the amount of the aqueous phase.

### (Production Method)

The oil phase components were mixed homogeneously. The aqueous phase components, which were mixed homogeneously in advance, were gently added thereto and stirred to obtain an emulsion. This emulsion was filled into a designated container to obtain a water-in-oil water-break cosmetic.

### [Evaluation of Stability of a Viscosity]

The stability of the resulting water-in-oil water-break cosmetic was evaluated based on the following evaluation criteria. The results are described in Tables 2, 3, and 4.

The resulting water-in-oil water-break cosmetic was filled into a 30-mL vial, and the viscosity of the cosmetic determined at 25° C. after storing the cosmetic in a thermostatic chamber at 50° C. for 7 days was compared with the viscosity of the cosmetic determined at 25° C. immediately after preparation, with a B-type viscometer (TVB-10; manufactured by Toki Sangyo Co., Ltd). The cosmetic whose change in viscosity, compared with the viscosity immediately after preparation, was 0% or more and less than 25% was evaluated as “A”. The cosmetic whose change in viscosity was 25% or more and less than 40% was evaluated as “B”. The cosmetic whose change in viscosity was 40% or more and less than 50% was evaluated as “C”. The cosmetic which was separated or whose change in viscosity was 50% or more was evaluated as “D”. Note that no characteristic evaluations were performed on formulations with a stability rating of D.

### [Evaluation of Usability]

The aesthetics (glossiness of the cosmetic's appearance), spread ability at the time of application, appearance at the finish (skin condition after application), stickiness at the finish, and durability (how long the makeup cosmetic lasts when layered) of the resulting water-in-oil water-break cosmetic was evaluated by 10 expert panelists based on the following evaluation criteria, and an average of the evaluation results was determined. Note that, for durability, the resulting water-in-oil water-break cosmetic was applied evenly, on which foundation was applied, and the condition after 3 hours was observed.

TABLE 5

| | | Spread | | | |
|---|---|---|---|---|---|
| | | ability at | Appearance | Stickiness | |
| | | the time of | of the | at the | |
| | Aesthetics | application | finish | finish | Durability |
| 5 points | Excellent | Excellent | Excellent | Not at all | Excellent |
| | | | | sticky | |
| 4 points | Very | Very | Very | Almost no | Very |
| | Good | Good | Good | sticky | Good |
| 3 points | Good | Good | Good | Good | Good |
| 2 points | Fair | Fair | Fair | Slightly | Fair |
| | | | | sticky | |
| 1 point | Poor | Poor | Poor | Strongly | Poor |
| | | | | sticky | |

The results based on the average scores of the evaluation results obtained are listed in Tables 2, 3 and 4 above according to the following criteria.
A: Average score is 4.5 or more
B: Average score is 3.5 or more to less than 4.5
C: Average score is 2.5 or more to less than 3.5
D: Average score is 1.5 or more to less than 2.5
F: Average score is less than 1.5

Cosmetics with a “C” rating or better were considered acceptable products.

As described in Tables 2, 3, and 4 above, the present cosmetic was remarkably excellent in terms of its aesthetics, spread ability at the time of application, appearance at its finish, stickiness of its finish, and durability, compared to those of Comparative Examples 1 to 6. Water-break cosmetics often have a matte appearance, but the present cosmetic exhibited glossiness and was excellent in terms of aesthetics. At the same time, the present cosmetic was also excellent in terms of its feeling when used and stability. On the other hand, Comparative Example 1, into which component (A) was not blended, could not be emulsified. Comparative Example 2, into which component (B) was not blended, could be emulsified but was a cosmetic having poor stability. Comparative Example 3, into which powder was not blended, and Comparative Examples 4 and 5, into which powder having low oil absorption efficiency was blended, were cosmetics having poor durability. Further, Comparative Example 6, in which component (C) was dispersed in the oil phase instead of the aqueous phase, did not exhibit glossiness of the cosmetic's appearance and had poor aesthetics.

Examples of the cosmetic are shown below. They were evaluated based on the same criteria as described above.

### Example 14

### W/O Eye Cream

### <Preparation of the Cosmetic>

A: Components 1 to 7 were homogeneously mixed.
B: Components 8 to 14 were homogeneously mixed.
C: The mixture obtained in step B was added to the mixture obtained in step A, and the resultant was emulsified to obtain W/O eye cream.

| Components | Mass (%) |
|---|---|
| 1. KSG-820 (Note 1) | 4 |
| 2. KSG-42A (Note 2) | 3 |
| 3. KF-6105 (Note 3) | 0.8 |
| 4. Simmondsia chinensis (jojoba) seed oil | 4 |
| 5. Limnanthes alba (meadowfoam) seed oil | 7 |
| 6. Butyrospermum parkii (shea) butter | 1 |
| 7. KP-550 (Note 4) | 2 |
| 8. Sodium hyaluronate | 0.1 |
| 9. Xylitol | 3 |
| 10. Sodium PCA | 2 |
| 11. Water dispersion (60%) of Synthesis Example 3 | 5 |
| 12. Pentylene glycol | 3 |
| 13. Sodium chloride | 0.5 |
| 14. Water | Remaining amount |
| Total | 100 |

(Note 1)
Mixture of 25% lauryl dimethicone/polyglycerin-3 crosspolymer + 75% isododecane (manufactured by Shin-Etsu Chemical Co., Ltd.)
(Note 2)
Mixture of 20% vinyl dimethicone/lauryl dimethicone crosspolymer + 80% isododecane (manufactured by Shin-Etsu Chemical Co., Ltd.)
(Note 3)
Lauryl polyglyceryl-3 polydimethylsiloxyethyl dimethicone (manufactured by Shin-Etsu Chemical Co., Ltd.)
(Note 4)
Acrylates/dimethicone copolymer (manufactured by Shin-Etsu Chemical Co., Ltd.)

The resulting W/O eye cream was excellent in terms of its aesthetics, spread ability at the time of application, appearance of its finish, stickiness of its finish, and durability.

### Example 15

### Hand Cream

### <Preparation of the Cosmetic>

A: Components 1 to 6 were homogeneously mixed.
B: Components 7 to 13 were homogeneously mixed.
C: The mixture obtained in step B was added to the mixture obtained in step A, and the resultant was emulsified to obtain hand cream.

| Components | Mass (%) |
|---|---|
| 1. KSG-310 (Note 1) | 3 |
| 2. KF-6048 (Note 2) | 0.2 |
| 3. KSG-44 (Note 3) | 1 |
| 4. Squalane | 8 |
| 5. Petrolatum | 2 |
| 6. KP-550 (Note 4) | 2 |
| 7. Butylene glycol | 8 |
| 8. Sodium citrate | 0.2 |
| 9. Magnesium sulfate | 0.3 |
| 10. Phenoxyethanol | 0.1 |
| 11. Methylparaben | 0.1 |
| 12. Water dispersion (51.4%) of Synthesis Example | 18 |
| 1 | |
| 13. Water | Remaining amount |
| Total | 100 |

(Note 1)
Mixture of mineral oil + 25% to 35% PEG-15/lauryl dimethicone crosspolymer (manufactured by Shin-Etsu Chemical Co., Ltd.)
(Note 2)
Cetyl PEG/PPG-10/1 dimethicone (manufactured by Shin-Etsu Chemical Co., Ltd.)
(Note 3)
Mixture of squalane + 25% to 35% vinyl dimethicone/lauryl dimethicone crosspolymer (manufactured by Shin-Etsu Chemical Co., Ltd.)
(Note 4)
Product obtained by dissolving 40% acrylates/dimethicone copolymer in isododecane (manufactured by Shin-Etsu Chemical Co., Ltd.)

The resulting hand cream was excellent in terms of its water-break properties, aesthetics, spread ability at the time of application, appearance of its finish, stickiness of its finish, and durability.

### Example 16

### Stick Foundation

### <Preparation of the Cosmetic>

A: Components 9 to 11 were dispersed with a three-roll mill.
B: Components 1 to 8 were heated to 95° C. and homogeneously mixed.
C: Components 12 to 16 and the mixture obtained in step A were homogeneously mixed and heated to 85° C.
D: The mixture obtained in step C was added to the mixture obtained in step B, and the resultant was emulsified and filled into a stick container, which was then gradually cooled, to obtain a stick foundation.

| Components | Mass (%) |
|---|---|
| 1. KSG-790 (Note 1) | 4 |
| 2. KF-6038 (Note 2) | 0.5 |
| 3. Stearoyl inulin (Note 3) | 2.5 |
| 4. Ceresin | 5.5 |
| 5. KF-56A (Note 4) | 11.5 |
| 6. KMP-590 (Note 5) | 1.5 |
| 7. Isotridecyl isononanoate | 4 |
| 8. KF-6105 (Note 6) | 0.3 |
| 9. KTP-09W (Note 7) | 6.5 |
| 10. KTP-09R, KTP-09Y, and KTP-09B (Note 7) | 1 |
| 11. Dipropylene glycol | 6 |
| 12. Water dispersion (60%) of Synthesis Example 3 | 1 |
| 13. Methylparaben | 0.1 |
| 14. Sodium citrate | 0.2 |
| 15. Sodium chloride | 0.5 |
| 16. Water | Remaining amount |
| Total | 100 |

(Note 1)
Mixture of caprylyl methicone + 25% to 35% dimethicone/polyglycerin-3 crosspolymer (manufactured by Shin-Etsu Chemical Co., Ltd.)
(Note 2)
Lauryl PEG-9 polydimethylsiloxyethyl dimethicone (manufactured by Shin-Etsu Chemical Co., Ltd.)
(Note 3)
Rheopearl ISK2 (manufactured by Chiba Flour Milling Co., Ltd.)
(Note 4)
Diphenylsiloxy phenyl trimethicone (manufactured by Shin-Etsu Chemical Co., Ltd.)
(Note 5)
Polymethylsilsesquioxane (manufactured by Shin-Etsu Chemical Co., Ltd.)
(Note 6)
Lauryl polyglyceryl-3 polydimethylsiloxyethyl dimethicone (manufactured by Shin-Etsu Chemical Co., Ltd.)
(Note 7)
Inorganic color pigments treated with KF-9909; W (white), R (red), Y (yellow), and B (black) (manufactured by Shin-Etsu Chemical Co., Ltd.)

The resulting stick foundation was excellent in terms of its water-break properties, aesthetics, spread ability at the time of application, appearance of its finish, stickiness of its finish, and durability.

### Example 17

### BB Cream

### <Preparation of the Cosmetic>

A: Components 9 to 14 were dispersed with a three-roll mill.
B: Components 1 to 8 and the mixture obtained in step A were homogeneously mixed.
C: Components 15 to 21 were homogeneously mixed.
D: The mixture obtained in step C was added to the mixture obtained in step B, and the resultant was emulsified to obtain BB cream.

| Components | Mass (%) |
|---|---|
| 1. KSG-330 (Note 1) | 3 |
| 2. KF-6038 (Note 2) | 0.2 |
| 3. KSG-18A (Note 3) | 5 |
| 4. KF-56A (Note 4) | 3 |
| 5. KF-7312J (Note 5) | 0.5 |
| 6. Ethylhexyl methoxycinnamate | 5 |
| 7. Diethylamino hydroxybenzoyl hexyl benzoate | 1 |
| 8. Octocrylene | 2 |
| 9. Ethylhexyl palmitate | 4 |
| 10. KP-578 (Note 6) | 0.5 |
| 11. KTP-09W (Note 7) | 5 |
| 12. KTP-09R, KTP-09Y, and KTP-09B (Note 7) | 1 |
| 13. Titanium oxide fine particles treated with lime | 2 |
| soap | |
| 14. Zinc oxide fine particles treated with silicone | 4 |
| 15. Glycerin | 8 |
| 16. Pentylene glycol | 1 |
| 17. Methylparaben | 0.1 |
| 18. Sodium citrate | 0.2 |
| 19. Sodium chloride | 0.5 |
| 20. Water dispersion (51.3%) of Synthesis | 10 |
| Example 2 | |
| 21. Water | Remaining amount |
| Total | 100 |

(Note 1)
Mixture of triethylhexanoin + 15% to 25% PEG-15/lauryl dimethicone crosspolymer (manufactured by Shin-Etsu Chemical Co., Ltd.)
(Note 2)
Lauryl PEG-9 polydimethylsiloxyethyl dimethicone (manufactured by Shin-Etsu Chemical Co., Ltd.)
(Note 3)
Mixture of diphenylsiloxy phenyl trimethicone + 10% to 20% dimethicone/phenyl vinyl dimethicone crosspolymer (manufactured by Shin-Etsu Chemical Co., Ltd.)
(Note 4)
Diphenylsiloxy phenyl trimethicone (manufactured by Shin-Etsu Chemical Co., Ltd.)
(Note 5)
Product obtained by dissolving 50% trimethylsiloxysilicate in cyclopentasiloxane (manufactured by Shin-Etsu Chemical Co., Ltd.)
(Note 6)
Acrylates/ethylhexyl acrylate/dimethicone methacrylate copolymer (manufactured by Shin-Etsu Chemical Co., Ltd.)
(Note 7)
Inorganic color pigments treated with KF-9909; W (white), R (red), Y (yellow), and B (black) (manufactured by Shin-Etsu Chemical Co., Ltd.)

The resulting BB cream was excellent in terms of its water-break properties, aesthetics, spread ability at the time of application, appearance of its finish, stickiness of its finish, and durability.

### Example 18

### Makeup Base Cream

### <Preparation of the Cosmetic>

A: Components 8 to 11 were dispersed with a three-roll mill.
B: Components 1 to 7 and the mixture obtained in step A were homogeneously mixed.
C: Components 12 to 18 were homogeneously mixed.
D: The mixture obtained in step C was added to the mixture obtained in step B, and the resultant was emulsified to obtain makeup base cream.

| Components | Mass (%) |
|---|---|
| 1. KSG-210 (Note 1) | 3.5 |
| 2. KF-6028 (Note 2) | 0.2 |
| 3. KSG-19 (Note 3) | 1 |
| 4. TMF-1.5 (Note 4) | 4.5 |
| 5. Ethylhexyl methoxycinnamate | 3 |
| 6. KP-549 (Note 5) | 1 |
| 7. KSP-441 (Note 6) | 2 |
| 8. Dimethicone (6 cs) | 5 |
| 9. KF-6106 (Note 7) | 0.6 |
| 10. Titanium oxide fine particles treated with lime | 4 |
| soap | |
| 11. Zinc oxide fine particles treated with silicone | 6 |
| 12. Glycerin | 3 |
| 13. Ethanol | 8 |
| 14. Phenoxyethanol | 0.3 |
| 15. Sodium citrate | 0.2 |
| 16. Sodium chloride | 1 |
| 17. Water dispersion (60%) of Synthesis Example 3, | 10 |
| 18. Water | Remaining amount |
| Total | 100 |

(Note 1)
Mixture of dimethicone + 20% to 30% dimethicone/PEG-10/15 crosspolymer (manufactured by Shin-Etsu Chemical Co., Ltd.)
(Note 2)
PEG-9 polydimethylsiloxyethyl dimethicone (manufactured by Shin-Etsu Chemical Co., Ltd.)
(Note 3)
Mixture of dimethicone + 10% to 20% dimethicone/vinyl dimethicone crosspolymer (manufactured by Shin-Etsu Chemical Co., Ltd.)
(Note 4)
Methyl trimethicone (manufactured by Shin-Etsu Chemical Co., Ltd.)
(Note 5)
Product obtained by dissolving 40% acrylates/dimethicone copolymer in methyl trimethicone (manufactured by Shin-Etsu Chemical Co., Ltd.)
(Note 6)
Polysilicone-22 (manufactured by Shin-Etsu Chemical Co., Ltd.)
(Note 7)
Polyglyceryl-3 polydimethylsiloxyethyl dimethicone (manufactured by Shin-Etsu Chemical Co., Ltd.)

The resulting makeup base cream was excellent in terms of its water-break properties, aesthetics, spread ability at the time of application, appearance of its finish, stickiness of its finish, and durability.

### Example 19

### W/O Concealer

### <Preparation of the Cosmetic>

A: Components 8 to 13 were dispersed with a three-roll mill.
B: Components 1 to 7 were homogeneously mixed.
C: Components 15 to 22 were homogeneously mixed.
D: The mixture obtained in step C was added to the mixture obtained in step B, and the resultant was emulsified. The mixture obtained in step A and component 14 were further added and mixed to obtain a W/O concealer.

| Components | Mass (%) |
|---|---|
| 1. KSG-270 (Note 1) | 5 |
| 2. KSG-18A (Note 2) | 1 |
| 3. KF-6038 (Note 3) | 0.5 |
| 4. Homosalate | 3.5 |
| 5. C13-15 alkane | 6.5 |
| 6. Cetyl ethylhexanoate | 3 |
| 7. Caprylic/capric triglyceride | 2 |
| 8. Isotridecyl isononanoate | 3 |
| 9. KP-578 (Note 4) | 0.5 |
| 10. KTP-09W (Note 5) | 13 |
| 11. KTP-09R (Note 5) | 0.7 |
| 12. KTP-09Y (Note 5) | 1.2 |
| 13. KTP-09B (Note 5) | 0.1 |
| 14. KMP-590 (Note 6) | 4 |
| 15. Sorbitol | 3 |
| 16. Pentylene glycol | 2 |
| 17. Butylene glycol | 5 |
| 18. Sodium citrate | 0.2 |
| 19. Magnesium sulfate | 1 |
| 20. KSP-441 (Component (C)) (Note 7) | 3 |
| 21. KF-6100 (Note 8) | 0.5 |
| 22. Water | Remaining amount |
| Total | 100 |

(Note 1)
Mixture of diphenylsiloxy phenyl trimethicone + 15% to 25% dimethicone/PEG-10/15 crosspolymer (manufactured by Shin-Etsu Chemical Co., Ltd.)
(Note 2)
Mixture of diphenylsiloxy phenyl trimethicone + 10% to 20% dimethicone/phenyl vinyl dimethicone crosspolymer (manufactured by Shin-Etsu Chemical Co., Ltd.)
(Note 3)
Lauryl PEG-9 polydimethylsiloxyethyl dimethicone (manufactured by Shin-Etsu Chemical Co., Ltd.)
(Note 4)
Acrylates/ethylhexyl acrylate/dimethicone methacrylate copolymer (manufactured by Shin-Etsu Chemical Co., Ltd.)
(Note 5)
Inorganic color pigments treated with KF-9909; W (white), R (red), Y (yellow), and B (black) (manufactured by Shin-Etsu Chemical Co., Ltd.)
(Note 6)
Polymethylsilsesquioxane (manufactured by Shin-Etsu Chemical Co., Ltd.)
(Note 7)
Polysilicone-22 (manufactured by Shin-Etsu Chemical Co., Ltd.)
(Note 8)
Polyglyceryl-3 disiloxane dimethicone (manufactured by Shin-Etsu Chemical Co., Ltd.)

The resulting W/O concealer was excellent in terms of its water-break properties, aesthetics, spread ability at the time of application, appearance of its finish, stickiness of its finish, and durability.

### Example 20

### W/O Blush

### <Preparation of the Cosmetic>

A: Components 7 to 14 were dispersed with a three-roll mill.
B: Components 1 to 6 were homogeneously mixed.
C: Components 16 to 22 were homogeneously mixed.
D: The mixture obtained in step C was added to the mixture obtained in step B, and the resultant was emulsified. The mixture obtained in step A and component 15 were further added to obtain a W/O blush.

| Components | Mass (%) |
|---|---|
| 1. KSG-270 (Note 1) | 5 |
| 2. KSG-18A (Note 2) | 2 |
| 3. KF-6038 (Note 3) | 0.1 |
| 4. Ethylhexyl methoxycinnamate | 5 |
| 5. KF-4422 (Note 4) | 2 |
| 6. KF-96L-2cs (Note 5) | 8 |
| 7. Mica | 0.3 |
| 8. Red 7 (Red 202) | 0.1 |
| 9. Yellow 5 (Yellow 4) | 0.3 |
| 10. Titanium oxide treated with KP-574 (Note 6) | 0.8 |
| 11. Iron oxide black treated with KP-574 (Note 6) | 0.06 |
| 12. Red 6 (Red 201) | 0.04 |
| 13. Iron oxide red treated with KP-574 (Note 6) | 0.2 |
| 14. Polyglyceryl-2 triisostearate | 1.2 |
| 15. KSP-441 (Note 7) | 1.5 |
| 16. Glycerin | 3 |
| 17. 1,2-Hexanediol | 2 |
| 18. Dipropylene glycol | 8 |
| 19. Sodium citrate | 0.2 |
| 20. Sodium chloride | 1 |
| 21. Water dispersion (60%) of Synthesis Example 3 | 5 |
| 22. Water | Remaining amount |
| Total | 100 |

(Note 1)
Mixture of diphenylsiloxy phenyl trimethicone + 15% to 25% dimethicone/PEG-10/15 crosspolymer (manufactured by Shin-Etsu Chemical Co., Ltd.)
(Note 2)
Mixture of diphenylsiloxy phenyl trimethicone + 10% to 20% dimethicone/phenyl vinyl dimethicone crosspolymer (manufactured by Shin-Etsu Chemical Co., Ltd.)
(Note 3)
Lauryl PEG-9 polydimethylsiloxyethyl dimethicone (manufactured by Shin-Etsu Chemical Co., Ltd.)
(Note 4)
Ethyl methicone (manufactured by Shin-Etsu Chemical Co., Ltd.)
(Note 5)
Dimethicone having a kinematic viscosity of 2 mm²/s at 25° C. (manufactured by Shin-Etsu Chemical Co., Ltd.)
(Note 6)
Acrylates/tridecyl acrylate/triethoxysilylpropyl methacrylate/dimethicone methacrylate copolymer (manufactured by Shin-Etsu Chemical Co., Ltd.)
(Note 7)
Polysilicone-22 (manufactured by Shin-Etsu Chemical Co., Ltd.)

The resulting W/O blush was excellent in terms of its water-break properties, aesthetics, spread ability at the time of application, appearance of its finish, stickiness of its finish, and durability.

### Example 21

### Hair Cream

### <Preparation of the Cosmetic>

A: Components 1 to 6 were homogeneously mixed.
B: Components 7 to 13 were homogeneously mixed.
C: The mixture obtained in step B was added to the mixture obtained in step A, and the resultant was emulsified to obtain hair cream.

| Components | Mass (%) |
|---|---|
| 1. KSG-210 (Note 1) | 3.5 |
| 2. KF-6017 (Note 2) | 0.2 |
| 3. KSG-18A (Note 3) | 1 |
| 4. TMF-1.5 (Note 4) | 3.5 |
| 5. Ethylhexyl methoxycinnamate | 3.5 |
| 6. KP-549 (Note 5) | 1 |
| 7. Glycerin | 3 |
| 8. Butylene glycol | 8 |
| 9. Phenoxyethanol | 0.3 |
| 10. Sodium citrate | 0.2 |
| 11. Sodium chloride | 1 |
| 12. Water dispersion (60%) of Synthesis Example 3 | 3 |
| 13. Water | Remaining amount |
| Total | 100 |

(Note 1)
Mixture of dimethicone + 20% to 30% dimethicone/PEG-10/15 crosspolymer (manufactured by Shin-Etsu Chemical Co., Ltd.)
(Note 2)
PEG-10 dimethicone (manufactured by Shin-Etsu Chemical Co., Ltd.)
(Note 3)
Mixture of diphenylsiloxy phenyl trimethicone + 10% to 20% dimethicone/phenyl vinyl dimethicone crosspolymer (manufactured by Shin-Etsu Chemical Co., Ltd.)
(Note 4)
Methyl trimethicone (manufactured by Shin-Etsu Chemical Co., Ltd.)
(Note 5)
Product obtained by dissolving 40% acrylates/dimethicone copolymer in methyl trimethicone (manufactured by Shin-Etsu Chemical Co., Ltd.)

The resulting hair cream was excellent in terms of its water-break properties, aesthetics, spread ability at the time of application, appearance of its finish, stickiness of its finish, and durability.

### Example 22

### W/O Cream

### <Preparation of the Cosmetic>

A: Components 1 to 5 were homogeneously mixed.
B: Components 6 to 11 were homogeneously mixed.
C: Under stirring, the mixture obtained in step B was gradually added to the mixture obtained in step A, and the resultant was emulsified to obtain W/O cream.

| Components | Mass (%) |
|---|---|
| 1. KSG-710 (Note 1) | 6 |
| 2. KSG-19 (Note 2) | 2 |
| 3. Dimethicone (6 cs) | 13.5 |
| 4. KF-4418 (Note 3) | 5 |
| 5. KF-6104 (Note 4) | 1 |
| 6. KSP-100W (Component (C)) (Note 5) | 2 |
| 7. Water dispersion (60%) of Synthesis Example 3 | 1 |
| 8. Sodium citrate | 0.2 |
| 9. Dipropylene glycol | 8 |
| 10. Diglycerin | 3 |
| 11. Water | Remaining amount |
| Total | 100 |

(Note 1)
Mixture of dimethicone (6 cs) + 20% to 30% dimethicone/polyglycerin-3 crosspolymer (manufactured by Shin-Etsu Chemical Co., Ltd.)
(Note 2)
Mixture of dimethicone (6 cs) + 10% to 20% dimethicone/vinyl dimethicone crosspolymer (manufactured by Shin-Etsu Chemical Co., Ltd.)
(Note 3)
Caprylyl methicone (manufactured by Shin-Etsu Chemical Co., Ltd.)
(Note 4)
Polyglyceryl-3 polydimethylsiloxyethyl dimethicone (manufactured by Shin-Etsu Chemical Co., Ltd.)
(Note 5)
Vinyl dimethicone/methicone silsesquioxane crosspolymer powder subjected to a hydrophilic treatment (manufactured by Shin-Etsu Chemical Co., Ltd.)

The resulting W/O cream was excellent in terms of its aesthetics, spread ability at the time of application, appearance of its finish, stickiness of its finish, and durability.

### Example 23

### W/O Cream Foundation

### <Preparation of the Cosmetic>

A: Components 1 to 6 were homogeneously mixed, components 7 to 10 were added thereto, and the resultant was mixed to be homogeneously.
B: Components 11 to 15 were homogeneously mixed.
C: Under stirring, the mixture obtained in step B was gradually added to the mixture obtained in step A, and the resultant was emulsified to obtain a W/O cream foundation.

| Components | Mass (%) |
|---|---|
| 1. KSG-45 (Note 1) | 2 |
| 2. KSG-790 (Note 2) | 2 |
| 3. Mineral oil | 2 |
| 4. Triethylhexanoin | 5 |
| 5. Isotridecyl isononanoate | 9 |
| 6. KF-6105 (Note 3) | 1 |
| 7. KMP-591 (Note 4) | 1 |
| 8. KSP-100 (Note 5) | 2 |
| 9. KTP-09W, KTP-09R, KTP-09Y, and KTP-09B | 20 |
| (Note 6) | |
| 10. KF-6115 (Note 7) | 0.5 |
| 11. Butylene glycol | 5 |
| 12. Sodium citrate | 1 |
| 13. Magnesium sulfate | 1 |
| 14. Water dispersion (60%) of Synthesis Example 3 | 4 |
| 15. Water | Remaining amount |
| Total | 100 |

(Note 1)
Mixture of coco-caprylate/caprate + 30% to 40% vinyl dimethicone/lauryl dimethicone crosspolymer (manufactured by Shin-Etsu Chemical Co., Ltd.)
(Note 2)
Mixture of caprylyl methicone + 25% to 35% dimethicone/polyglycerin-3 crosspolymer (manufactured by Shin-Etsu Chemical Co., Ltd.)
(Note 3)
Lauryl polyglyceryl-3 polydimethylsiloxyethyl dimethicone (manufactured by Shin-Etsu Chemical Co., Ltd.)
(Note 4)
Polymethylsilsesquioxane (manufactured by Shin-Etsu Chemical Co., Ltd.)
(Note 5)
Vinyl dimethicone/methicone silsesquioxane crosspolymer (manufactured by Shin-Etsu Chemical Co., Ltd.)
(Note 6)
Inorganic color pigments treated with KF-9909; W (white), R (red), Y (yellow), and B (black) (manufactured by Shin-Etsu Chemical Co., Ltd.)
(Note 7)
Lauryl polyglyceryl-3 polydimethylsiloxyethyl dimethicone (manufactured by Shin-Etsu Chemical Co., Ltd.)

The resulting W/O cream foundation was excellent in terms of its aesthetics, spread ability at the time of application, appearance of its finish, stickiness of its finish, and durability.

### Example 24

### Sunscreen Milky Lotion

### <Preparation of the Cosmetic>

A: Components 1 to 10 were homogeneously mixed.
B: Components 13 to 18 were homogeneously mixed.
C: The mixture obtained in step B was added to the mixture obtained in step A, and the resultant was homogenously mixed.
D: Components 11 and 12 were added to the mixture obtained in step C, and the resultant was homogeneously mixed.
E: The mixture obtained in step D was degassed and then filled into a container to obtain a sunscreen milky lotion.

| Components | Mass (%) |
|---|---|
| 1. KSG-270 (Note 1) | 3 |
| 2. KSG-18A (Note 2) | 3 |
| 3. KF-6048 (Note 3) | 0.3 |
| 4. KF-56A (Note 4) | 5 |
| 5. KF-4422 (Note 5) | Remaining amount |
| 6. Ethylhexyl methoxycinnamate | 5 |
| 7. Ethylhexyl salicylate | 2 |
| 8. Octocrylene | 1 |
| 9. Diethylamino hydroxybenzoyl hexyl benzoate | 2 |
| 10. KSP-105 (Note 6) | 2 |
| 11. SPD-T7 (Note 7) | 10 |
| 12. SPD-Z5 (Note 8) | 10 |
| 13. Butylene glycol | 3 |
| 14. Ethanol | 5 |
| 15. Sodium citrate | 0.2 |
| 16. Sodium chloride | 0.5 |
| 17. KSP-100W (Component (C)) (Note 9) | 3 |
| 18. Water | 17 |
| Total | 100 |

(Note 1)
Mixture of diphenylsiloxy phenyl trimethicone + 15% to 25% dimethicone/PEG-10/15 crosspolymer (manufactured by Shin-Etsu Chemical Co., Ltd.)
(Note 2)
Mixture of diphenylsiloxy phenyl trimethicone + 10% to 20% dimethicone/phenyl vinyl dimethicone crosspolymer (manufactured by Shin-Etsu Chemical Co., Ltd.)
(Note 3)
Cetyl PEG/PPG-10/1 dimethicone (manufactured by Shin-Etsu Chemical Co., Ltd.)
(Note 4)
Diphenylsiloxy phenyl trimethicone (manufactured by Shin-Etsu Chemical Co., Ltd.)
(Note 5)
Ethyl methicone (manufactured by Shin-Etsu Chemical Co., Ltd.)
(Note 6)
Vinyl dimethicone/methicone silsesquioxane crosspolymer (manufactured by Shin-Etsu Chemical Co., Ltd.)
(Note 7)
Product obtained by dispersing 45% titanium oxide fine particles in cyclopentasiloxane (manufactured by Shin-Etsu Chemical Co., Ltd.)
(Note 8)
Product obtained by dispersing 60% zinc oxide fine particles in cyclopentasiloxane (manufactured by Shin-Etsu Chemical Co., Ltd.)
(Note 9)
Vinyl dimethicone/methicone silsesquioxane crosspolymer powder subjected to a hydrophilic treatment (manufactured by Shin-Etsu Chemical Co., Ltd.)

The resulting sunscreen milky lotion was excellent in terms of its aesthetics, spread ability at the time of application, appearance of its finish, stickiness of its finish, and durability.

### Example 25

### O/W/O Sunscreen Cream

### <Preparation of the Cosmetic>

A: Components 1 to 8 were homogeneously mixed.
B: Components 15 and 16 were homogeneously mixed.
C: Components 9 to 12 were homogeneously dissolved in component 13, and component 14 was swelled with the resultant, then mixed with B.
D: Under stirring, the mixture obtained in step C was gradually added to the mixture obtained in step A, and the resultant was emulsified to obtain an O/W/O sunscreen cream.

| Components | Mass (%) |
|---|---|
| 1. C9-12 alkane | 10 |
| 2. KSG-320 (Note 1) | 3.5 |
| 3. KSG-1510 (Note 2) | 3 |
| 4. KF-6180 (Note 3) | 0.5 |
| 5. Bis-ethylhexyloxyphenol methoxyphenyl triazine | 1.5 |
| 6. Homosalate | 7.5 |
| 7. Butyl methoxydibenzoylmethane | 3 |
| 8. Ethylhexyl salicylate | 3 |
| 9. Butylene glycol | 5 |
| 10. Sodium citrate | 0.5 |
| 11. Sodium chloride | 1 |
| 12. Preservative | 0.3 |
| 13. Water | Remaining |
| | amount |
| 14. Ammonium acryloyldimethyltaurate/VP copolymer | 0.1 |
| 15. KSP-100W | 2 |
| 16. Dimethicone (2 cs) | 1.5 |
| Total | 100 |

(Note 1)
Mixture of 70% to 80% isododecane + 20% to 30% PEG-15/lauryl dimethicone crosspolymer (manufactured by Shin-Etsu Chemical Co., Ltd.)
(Note 2)
Mixture of 90% to 95% methyl trimethicone + 5% to 10% dimethicone/vinyl dimethicone crosspolymer (manufactured by Shin-Etsu Chemical 0Co., Ltd.)
(Note 3)
Isostearyl polyglyceryl-3 dimethicone (manufactured by Shin-Etsu Chemical Co., Ltd.)

The resulting O/W/O sunscreen cream was excellent in terms of its aesthetics, spread ability at the time of application, appearance of its finish, stickiness of its finish, and durability. Note that the components of the external oil phase and the internal oil phase of the O/W/O sunscreen cream have poor compatibility, but they are stably blended in the present cosmetic, resulting in a feeling when used that has not been achieved with conventional products.

1. A water-in-oil cosmetic comprising an oil agent and the following components (A), (B) and (C), wherein the following component (C) is contained in an aqueous phase component of the water-in-oil cosmetic,
(A) one or more kinds of silicone selected from a partially crosslinked polyether-modified silicone and a partially crosslinked polyglycerin-modified silicone in an amount of 0.1 to 4% by mass in the water-in-oil cosmetic,
(B) a non-crosslinked silicone surfactant in an amount of 0.05% to 1.5% by mass in the water-in-oil cosmetic, and
(C) a silicone powder absorbing 30 parts by mass or more of squalane or ethylhexyl palmitate relative to 100 parts by mass of the silicone powder in an amount of 0.5% to 10% by mass in the water-in-oil cosmetic.

2. The water-in-oil cosmetic according to claim 1, wherein an amount of the aqueous phase component comprising component (C) is 30 to 90% by mass in the water-in-oil cosmetic.

3. The water-in-oil cosmetic according to claim 1, wherein component (C) is dispersed in an internal aqueous phase of the water-in-oil cosmetic.

4. The water-in-oil cosmetic according to claim 1, wherein the silicone powder absorbs 30 parts by mass or more of squalane or ethylhexyl palmitate relative to 100 parts by mass of the silicone powder by an immersion of the silicone powder in squalane or ethylhexyl palmitate at 25° C. for 24 hours.

5. The water-in-oil cosmetic according to claim 1, wherein the silicone powder is selected from the group consisting of silicone rubber powder coated with a silicone resin and silicone rubber particles.

6. The water-in-oil cosmetic according to claim 1, wherein the silicone powder is a silicone rubber particle of an addition reaction product of (C-1) an organohydrogen polysiloxane and (C-2) an alkenyl group containing-organopolysiloxane,
wherein one or more selected from components (C-1) and (C-2) are a silicone rubber particle having 5 to 70 mol % of alkyl groups having 6 to 30 carbon atoms bonded to silicon atoms, based on the total mole of all substituents bonded to the silicon atoms.

7. The water-in-oil cosmetic according to claim 6, wherein component (C-1) in the silicone rubber particle has 5 to 70 mol % of alkyl groups having 6 to 30 carbon atoms bonded to silicon atoms, based on the total mole of all substituents bonded to the silicon atoms.

8. The water-in-oil cosmetic according to claim 6, wherein
component (C-1) is an organohydrogen polysiloxane having two or more hydrogen atoms each bonded to a silicon atom in one molecule, as represented by the following formula (1): 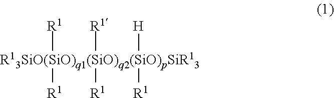
wherein R¹ is, independently of each other, a group selected from an alkyl group having 1 to 5 carbon atoms, an aryl group having 6 to 20 carbon atoms, and an aralkyl group having 7 to 20 carbon atoms, R^{1′} is an alkyl group having 6 to 30 carbon atoms, q1 and q2 are, independently of each other, an integer of 1 to 60, p is an integer of 2 to 20, and the parenthesized siloxane units, p, q1 and q2, may form a block unit or be sequenced at random, and
component (C-2) is an organopolysiloxane having two or more alkenyl groups in one molecule, as represented by the following formula (2): 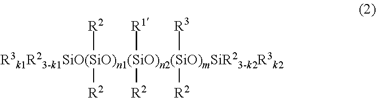
wherein R^{1′} is an alkyl group having 6 to 30 carbon atoms, R² is, independently of each other, a group selected from an alkyl group having 1 to 5 carbon atoms an aryl group having 6 to 20 carbon atoms, and an aralkyl group having 7 to 20 carbon atoms, R³ is an alkenyl group having 2 to 6 carbon atoms, n1 is an integer of 1 to 40, n2 is an integer of 0 to 50, m is an integer of 0 to 10, k1 and k2 are each an integer of 0 to 3, m+k1+k2 is an integer of 2 or more, and the parenthesized siloxane units, m, n1 and n2, may form a block unit or be sequenced at random, wherein an amount of component (C-2) is such that a mole ratio of the alkenyl group in component (C-2) is 0.5 to 2, per mole of the hydrosilyl group in component (C-1).

9. The water-in-oil cosmetic according to claim 6, wherein the silicone rubber particle further comprises a platinum group metal catalyst (C-3) in addition to components (C-1) and (C-2).

10. The water-in-oil cosmetic according to claim 6, comprising a water dispersion of the silicone rubber particles according to claim 6, wherein the water dispersion comprises
5 to 80% by mass of the silicone rubber particles,
0.01 to 15% by mass of a surfactant and
19 to 94% by mass of water.

11. The water-in-oil cosmetic according to claim 1, wherein silicone powder is spherical silicone rubber particles having a volume average particle size of 0.1 to 100 μm.

12. The water-in-oil cosmetic according to claim 5, wherein the silicone rubber particles are a vinyl dimethicone/lauryl dimethicone crosspolymer as defined in the cosmetic labeling name.

13. The water-in-oil cosmetic according to claim 5, wherein the silicone rubber powder coated with a silicone resin is at least one selected from the group consisting of vinyl dimethicone/methicone silsesquioxane crosspolymer, diphenyl dimethicone/vinyl diphenyl dimethicone/silsesquioxane crosspolymer, polysilicone-22, and polysilicone-1 crosspolymer, as defined in the cosmetic labeling name.

14. The water-in-oil cosmetic according to claim 5, wherein the silicone rubber powder coated with a silicone resin is vinyl dimethicone/methicone silsesquioxane crosspolymer as defined in the cosmetic labeling name, wherein the silicone rubber powder is coated with a silicone resin and has a hydrophilic treated surface.

15. The water-in-oil cosmetic according to claim 1, comprising (E) a volatile oil agent as the oil agent in an amount of 1 to 95% by mass in the water-in-oil cosmetic.

16. The water-in-oil cosmetic according to claim 1, comprising (F) a partially crosslinked organopolysiloxane having siloxane chain as a crosslinked segment in an amount of 0.01 to 30% by mass in the water-in-oil cosmetic.

17. The water-in-oil cosmetic according to claim 1, wherein the water-in-oil cosmetic is a water-break type.

18. The water-in-oil cosmetic according to claim 1, wherein the water-in-oil cosmetic is a foundation.

19. A method for preparing a water-in-oil cosmetic comprising the steps of adding an aqueous phase component comprising the following components (C) and (D) into an oil phase component comprising an oil agent and the following components (A) and (B), and then stirring to obtain an emulsion
(A) one or more kinds of silicone selected from a partially crosslinked polyether-modified silicone and a partially crosslinked polyglycerin-modified silicone in an amount of 0.1 to 4% by mass in the water-in-oil cosmetic,
(B) a non-crosslinked silicone surfactant in an amount of 0.05% to 1.5% by mass in the water-in-oil cosmetic, and
(C) a silicone powder absorbing 30 parts by mass or more of squalane or ethylhexyl palmitate relative to 100 parts by mass of the silicone powder in an amount of 0.5 to 10% by mass in the water-in-oil cosmetic,
(D) an aqueous component in an amount such that a total amount of the aqueous phase component including component (C) is 30 to 90% by mass in the water-in-oil cosmetic.

20. The water-in-oil cosmetic according to claim 7, comprising a water dispersion of the silicone rubber particles according to claim 7, wherein the water dispersion comprises
5 to 80% by mass of the silicone rubber particles,
0.01 to 15% by mass of a surfactant and
19 to 94% by mass of water.
